SEQUENCE LISTING (13 sequences)

SEQ ID NO: 1 - protein, 340 aa, synthetic construct
  REGION 1..340: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHYQVSFCLVMVLLFAVDTGLYFSVKTNIRSSTRDWKDHKFKWRKDPQDK

SEQ ID NO: 2 - protein, 336 aa, synthetic construct
  REGION 1..336: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLFFPPGYQVSFCLVMVLLFAVDTGLYFSVKTNIRSSTRDWKDHKFKWRKDPQDK

SEQ ID NO: 3 - protein, 335 aa, synthetic construct
  REGION 1..335: note = Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 4 - protein, 379 aa, synthetic construct
  REGION 1..379: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQ

SEQ ID NO: 5 - protein, 375 aa, synthetic construct
  REGION 1..375: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYL

SEQ ID NO: 6 - protein, 242 aa, synthetic construct
  REGION 1..242: note = Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIR

SEQ ID NO: 7 - protein, 758 aa, synthetic construct
MDFQVQIFSFLLISASVIMSREVQLVESGGGLVQPGGSLRLSCAASGFTFSRYWFSWVRQAPGKGLVWVGEINPSSSTINYAPSLKDKFTISRDNAKNTLYLQMNSLRAEDTAVYYCASLYYDYGDAYDYWGQGTLVTVSSGSTSGSGKPGSGEGSTKGEIVMTQSPATLSVSPGERATLSCKASQSVESNVAWYQQKPGQAPRALIYSASLRFSGIPARFSGSGSGTEFTLTISSLQSEDFAVYYCQQYNNYPLTFGAGTKLELKESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 8 aa, synthetic construct
GFTFSRYW

SEQ ID NO: 9 - protein, 8 aa, synthetic construct
INPSSSTI

SEQ ID NO: 10 - protein, 13 aa, synthetic construct
ASLYYDYGDAYDY

SEQ ID NO: 11 - protein, 6 aa, synthetic construct
QSVESN

SEQ ID NO: 12 - skipped

SEQ ID NO: 13 - protein, 9 aa, synthetic construct
QQYNNYPLT

(19) United States
(12) Patent Application Publication
(10) Pub. No.: US 2024/0382523 A1
(43) Pub. Date: Nov. 21, 2024

(54) OFF-THE-SHELF IPSC-DERIVED CAR-NK CELL AS MONOTHERAPY AND IN COMBINATION WITH ANTIBODY

(71) Applicant: Fate Therapeutics, Inc., San Diego, CA (US)

(72) Inventors: Bahram VALAMEHR, San Diego, CA (US); Yu-Waye CHU, San Diego, CA (US)

(21) Appl. No.: 18/684,574

(22) PCT Filed: Aug. 18, 2022
(86) PCT No.: PCT/US2022/075181

§ 371 Date: Feb. 16, 2024

Related U.S. Application Data
(60) Provisional application No. 63/234,656, filed on Aug. 18, 2021.

(51) Int. Cl. A61K 35/17; A61K 39/00; C07K 16/28; C12N 5/0783

(52) U.S. Cl. CPC A61K 35/17; A61K 39/4613; A61K 39/4631; A61K 39/464417; C07K 16/2878; C07K 16/2896; C12N 5/0646; A61K 2239/13; A61K 2239/21; A61K 2239/38; A61K 2239/48; C07K 2317/565; C07K 2317/622; C07K 2317/732; C07K 2319/03; C12N 2510/00

(57) ABSTRACT

Provided are methods and compositions for use in cancer immunotherapies. Exemplary compositions include functionally enhanced derivative effector cells obtained from directed differentiation of genomically engineered iPSCs. In some embodiments, derivative cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATION

This application claims priority to U.S. Provisional Application Ser. No. 63/234,656, filed Aug. 18, 2021, the disclosure of which is hereby incorporated by reference in its entirety.

### REFERENCE TO SEQUENCE LISTING SUBMITTED ELECTRONICALLY

The Sequence Listing titled 184143-636601_SL.xml, which was created on Aug. 18, 2022 and is 16,431 bytes in size, is hereby incorporated by reference in its entirety.

### FIELD OF THE INVENTION

The present disclosure is broadly concerned with the field of off-the-shelf immunocellular products. More particularly, the present disclosure is concerned with the strategies for developing multifunctional effector cells capable of delivering therapeutically relevant properties in vivo. The cell products developed under the present disclosure address critical limitations of patient-sourced cell therapies.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit. There is also the need to improve the efficacy and persistence of adoptively transferred lymphocytes to promote favorable patient outcome. Lymphocytes such as T cells and natural killer (NK) cells are potent anti-tumor effectors that play an important role in innate and adaptive immunity. However, the use of these immune cells for adoptive cell therapies remain to be challenging and have unmet needs for improvement. Therefore, significant opportunities remain to harness the full potential of T and NK cells, or other lymphocytes in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of embodiments of the present invention to provide methods and compositions for adoptive cell therapy, wherein the adoptive cell therapy includes administering an adoptive cell therapy product generated from derivative non-pluripotent cells differentiated from a single cell derived iPSC (induced pluripotent stem cell) clonal line, which iPSC line comprises one or several genetic modifications in its genome. Said one or several genetic modifications include, in some embodiments, one or more of DNA insertion, deletion, and substitution, and which modifications are retained and remain functional in subsequently derived cells after differentiation, expansion, passaging and/or transplantation.

The iPSC-derived non-pluripotent cells of the present application include, but are not limited to, CD34⁺ cells, hemogenic endothelium cells, HSCs (hematopoietic stem and progenitor cells), hematopoietic multipotent progenitor cells, T cell progenitors, NK cell progenitors, T cells, NKT cells, NK cells, and B cells. The iPSC-derived non-pluripotent cells of the present application comprise one or several genetic modifications in their genome through differentiation from an iPSC comprising the same genetic modifications. In some embodiments, the engineered clonal iPSC differentiation strategy for obtaining genetically engineered derivative cells benefits from a developmental potential of the iPSC in a directed differentiation that is not significantly adversely impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Accordingly, in one aspect, the present invention provides a method of treating a subject suitable for an adoptive cell therapy, (i) wherein the subject has a hematological cancer; (ii) wherein the method comprises administering to the subject at least a first cycle of an adoptive cell therapy product, with the first cycle comprising one or more doses of the adoptive cell therapy product administered in a first effective amount at a preselected frequency, and with an option of administering one or more additional cycles with one or more doses in a second effective amount, during a course of treatment over a period of time; (iii) wherein the first and the second effective amounts are the same or different; and (iv) wherein the product comprises an engineered natural killer (NK) lineage cell comprising exogenous CD16 expression, IL15RF expression, CD38 knockout and a BCMA directed CAR (chimeric antigen receptor). In various embodiments, the hematological cancer comprises: (i) multiple myeloma (MM); or (ii) relapsed or refractory MM (r/r MM). In some embodiments of the method, the course of treatment further comprises administering to the subject an effective amount of a tumor-targeting, ADCC-capable monoclonal antibody (mAb).

In some embodiments of the method, the course of treatment further comprises administering to the subject initial doses of a monoclonal antibody in an effective amount at a starting time prior to the first cycle of administering the adoptive cell therapy product, wherein the monoclonal antibody is an anti-CD38 monoclonal antibody. In various embodiments of the method of treating, the starting time is about 8-12 days prior to the first cycle of administering the adoptive cell therapy product, and wherein the initial doses of the monoclonal antibody comprise 6-10 weekly (QW) doses, and optionally followed by 6-10 bi-weekly (Q2W±1 day) doses thereafter. In some embodiments, the course of treatment further comprises administering to the subject the same anti-CD38 monoclonal antibody in an effective amount for 8 bi-weekly doses (Q2W±1 day) following the initial doses of administration of the monoclonal antibody. In some embodiments, the course of treatment further comprises administering to the subject one dose of the same anti-CD38 monoclonal antibody every four weeks (Q4W±1 day) in an effective amount until dose termination. In some embodiments, the anti-CD38 monoclonal antibody comprises daratumumab. In some embodiments, the course of treatment comprises administering to the subject an anti-CD38 monoclonal antibody, and wherein the method: (i) does not require lympho-conditioning; or (ii) requires a minimal need of lympho-conditioning. In some embodiments, the lympho-conditioning is CY/FLU-based.

In various embodiments of the method of treating, the method further comprises administering to the subject at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time. In some embodiments, the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m². In some embodiments, the duration between is: (i) about 40-84 hours; or (ii) about 3 days.

In various embodiments of the method of treating, the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR. In some embodiments, the effective amount of the adoptive cell therapy product in a dose is about 5×10⁷ cells to about 3×10⁹ cells. In some embodiments, the effective amount of the adoptive cell therapy product in each dose is about 1×10⁸, 3×10⁸, 10×10⁸, or about 1.5×10⁹ cells. In some embodiments, (i) daratumumab is in an amount of about 15 mg/kg to about 17 mg/kg; or (ii) daratumumab is in an amount of about 16 mg/kg.

In various embodiments of the method of treating, the subject suitable for the adoptive cell therapy product has a diagnosis of MM comprising: (i) a measurable disease; or (ii) having no complete remission (CR), or having relapse, or evidence of progressive disease (PD) after one or more prior lines of therapy. In some embodiments, the prior lines of therapy for MM, or relapsed or refractory MM comprise: (a) a chemotherapy, an immunochemotherapy, hematopoietic stem cell transplantation, chimeric antigen receptor (CAR) T-cell therapy, or any combination thereof, or (b) a proteasome inhibitor, an anti-CD38 antibody, an anti-SLAMF7 antibody, an immunomodulatory drug, a stem-cell transplantation (SCT), or any combination thereof; or (c) bortezomib, carfilzomib, ixazomib, daratumumab, isatuximab, elotuzumab, thalidomide, lenalidomide, pomalidomide, or any combination thereof, or (d) Carfilzomib.

In various embodiments of the method of treating, the method comprises administering (i) one cycle of the adoptive cell therapy product over about 29 days with 1 or 2 doses per cycle; (ii) two or more cycles of the adoptive cell therapy product, with each cycle comprising 1 or 2 doses; (iii) single dose per cycle over about 29 days in one cycle, with one or more cycles of the adoptive cell therapy product over an extended period of time based on clinical assessment of disease response; or (iv) 2 doses in one cycle over about 29 days, with one or more cycles of the adoptive cell therapy product over an extended period of time based on clinical assessment of disease response. In various embodiments of the method of treating, the administering of the adoptive cell therapy product is (i) via intravenous infusion, and/or (ii) at a site of an outpatient setting; and/or wherein each dose of the adoptive cell therapy product is cryopreserved, and then thawed prior to administering. In various embodiments of the method of treating, the BCMA directed CAR comprises: (i) a variable heavy chain (VH) and a variable light chain (VL), wherein: (a) said VH comprises: a heavy chain complementary determining region 1 (H-CDR1) with at least 80% sequence identity (e.g., 90% or 100% identity) to SEQ ID NO: 8 (GFTFSRYW), a heavy chain complementary determining region 2 (H-CDR2) with at least 80% sequence identity (e.g., 90% or 100% identity) to SEQ ID NO: 9 (INPSSSTI), and a heavy chain complementary determining region 3 (H-CDR3) with at least 80% sequence identity (e.g., 90% or 100% identity) to SEQ ID NO: 10 (ASLYYDYGDAYDY); and (b) a variable light chain (VL), said VL comprising: a light chain complementary determining region 1 (L-CDR1) with at least 80% sequence identity (e.g., 90% or 100% identity) to SEQ ID NO: 11 (QSVESN), a light chain complementary determining region 2 (L-CDR2) with at least 80% sequence identity (e.g., 90% or 100% identity) to SEQ ID NO: 12 (SAS), and a light chain complementary determining region 3 (L-CDR3) with at least 80% sequence identity (e.g., 90% or 100% identity) to SEQ ID NO: 13 (QQYNNYPLT); or (ii) an amino acid sequence that is of at least about 99%, 98%, 96%, 95%, 90%, 85%, or 80% identity to SEQ ID NO: 7. In some embodiments, the BCMA directed CAR comprises the amino acid sequence of SEQ ID NO: 7.

In another aspect, the present invention provides a composition comprising an engineered natural killer (NK) lineage cell for use in treating a hematological cancer in a subject, (i) wherein said engineered NK lineage cell comprises exogenous CD16 expression, IL15RF expression, CD38 knockout and a BCMA directed CAR (chimeric antigen receptor); (ii) wherein said use comprises a course of treatment comprising at least a first cycle of an adoptive cell therapy product comprising said engineered NK lineage cell, with the first cycle comprising one or more doses of the adoptive cell therapy product in a first effective amount at a preselected frequency, and with an option of one or more additional cycles with one or more doses in a second effective amount, over a period of time; and (iii) wherein the first and the second effective amounts are the same or different. In various embodiments, the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR. In some embodiments, the course of treatment further comprises an effective amount of a tumor-targeting, ADCC-capable monoclonal antibody (mAb). In some embodiments, the course of treatment further comprises initial doses of a monoclonal antibody provided in an effective amount at a starting time prior to the first cycle of the adoptive cell therapy product, wherein the monoclonal antibody is an anti-CD38 monoclonal antibody. In some embodiments, the anti-CD38 monoclonal antibody comprises daratumumab. In some embodiments, the course of treatment further comprises at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time. In some embodiments, the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m². In some embodiments, the BCMA directed CAR comprises: (i) a variable heavy chain (VH) and a variable light chain (VL), wherein: (a) said VH comprises: a heavy chain complementary determining region 1 (H-CDR1) with at least 80% sequence identity (e.g., 90% or 100% identity) to SEQ ID NO: 8 (GFTFSRYW), a heavy chain complementary determining region 2 (H-CDR2) with at least 80% sequence identity (e.g., 90% or 100% identity) to SEQ ID NO: 9 (INPSSSTI), and a heavy chain complementary determining region 3 (H-CDR3) with at least 80% sequence identity (e.g., 90% or 100% identity) to SEQ ID NO: 10 (ASLYYDYGDAYDY); and (b) a variable light chain (VL), said VL comprising: a light chain complementary determining region 1 (L-CDR1) with at least 80% sequence identity (e.g., 90% or 100% identity) to SEQ ID NO: 11 (QSVESN), a light chain complementary determining region 2 (L-CDR2) with at least 80% sequence identity (e.g., 90% or 100% identity) to SEQ ID NO: 12 (SAS), and a light chain complementary determining region 3 (L-CDR3) with at least 80% sequence identity (e.g., 90% or 100% identity) to SEQ ID NO: 13 (QQYNNYPLT); or (ii) an amino acid sequence that is of at least about 99%, 98%, 96%, 95%, 90%, 85%, or 80% identity to SEQ ID NO: 7. In some embodiments, the BCMA directed CAR comprises the amino acid sequence of SEQ ID NO: 7.

In another aspect, the present invention provides for use of an engineered natural killer (NK) lineage cell in the manufacture of an adoptive cell therapy product for treating a hematological cancer, (i) wherein said engineered NK lineage cell comprises exogenous CD16 expression, IL15RF expression, CD38 knockout and a BCMA directed CAR (chimeric antigen receptor); (ii) wherein said adoptive cell therapy product is for use in a course of treatment comprising at least a first cycle of the adoptive cell therapy product, with the first cycle comprising one or more doses of the adoptive cell therapy product in a first effective amount at a preselected frequency, and with an option of one or more additional cycles with one or more doses in a second effective amount, over a period of time; and (iii) wherein the first and the second effective amounts are the same or different. In various embodiments, the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR. In some embodiments, the course of treatment further comprises an effective amount of a tumor-targeting, ADCC-capable monoclonal antibody (mAb). In some embodiments, the course of treatment further comprises initial doses of a monoclonal antibody provided in an effective amount at a starting time prior to the first cycle of the adoptive cell therapy product, wherein the monoclonal antibody is an anti-CD38 monoclonal antibody. In some embodiments, the anti-CD38 monoclonal antibody comprises daratumumab. In some embodiments, the course of treatment further comprises at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time. In some embodiments, the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m². In some embodiments, the BCMA directed CAR comprises: (i) a variable heavy chain (VH) and a variable light chain (VL), wherein: (a) said VH comprises: a heavy chain complementary determining region 1 (H-CDR1) with at least 80% sequence identity (e.g., 90% or 100% identity) to SEQ ID NO: 8 (GFTFSRYW), a heavy chain complementary determining region 2 (H-CDR2) with at least 80% sequence identity (e.g., 90% or 100% identity) to SEQ ID NO: 9 (INPSSSTI), and a heavy chain complementary determining region 3 (H-CDR3) with at least 80% sequence identity (e.g., 90% or 100% identity) to SEQ ID NO: 10 (ASLYYDYGDAYDY); and (b) a variable light chain (VL), said VL comprising: a light chain complementary determining region 1 (L-CDR1) with at least 80% sequence identity (e.g., 90% or 100% identity) to SEQ ID NO: 11 (QSVESN), a light chain complementary determining region 2 (L-CDR2) with at least 80% sequence identity (e.g., 90% or 100% identity) to SEQ ID NO: 12 (SAS), and a light chain complementary determining region 3 (L-CDR3) with at least 80% sequence identity (e.g., 90% or 100% identity) to SEQ ID NO: 13 (QQYNNYPLT); or (ii) an amino acid sequence that is of at least about 99%, 98%, 96%, 95%, 90%, 85%, or 80% identity to SEQ ID NO: 7. In some embodiments, the BCMA directed CAR comprises the amino acid sequence of SEQ ID NO: 7.

In another aspect, the present invention provides a method of treating a subject diagnosed with multiple myeloma (MM), wherein the method comprises: (i) administering to the subject at least a first cycle of an adoptive cell therapy product, with the first cycle comprising at least a first dose at a first frequency of the adoptive cell therapy product administered in a first effective amount, and with an option of administering one or more additional cycles with one or more additional doses at the same first frequency or a different frequency in a second effective amount, during a course of treatment over a period of time; and (ii) administering to the subject weekly doses of an effective amount of an anti-CD38 monoclonal antibody or an anti-SLAMF7 monoclonal antibody for about 6-10 weeks, wherein a first weekly dose of the anti-CD38 monoclonal antibody or the anti-SLAMF7 monoclonal antibody precedes step (i) by about 8 to 12 days; wherein the MM tumor progress or relapse is prevented or reduced in the subject after the treatment using the adoptive cell therapy product; and wherein the adoptive cell therapy comprises engineered natural killer (NK) lineage cell comprising CD38 knockout, expressing an exogenous CD16, an IL15RF and a BCMA directed CAR. In various embodiments, the subject (i) has a refractory or a relapsed MM, and/or (ii) has been treated with one or more lines of therapy for MM. comprising: (a) a chemotherapy, an immunochemotherapy, hematopoietic stem cell transplantation, chimeric antigen receptor (CAR) T-cell therapy, or any combination thereof; (b) a proteasome inhibitor, an anti-CD38 antibody, an anti-SLAMF7 antibody, an immunomodulatory drug, a stem-cell transplantation (SCT), or any combination thereof, or (c) bortezomib, carfilzomib, ixazomib, daratumumab, isatuximab, elotuzumab, thalidomide, lenalidomide, pomalidomide, or any combination thereof.

In various embodiments of the method of treating, the step (ii) further comprises administering bi-weekly doses of an effective amount of the same anti-CD38 monoclonal antibody or anti-SLAMF7 monoclonal antibody for another 6-10 weeks after completing the weekly doses. In some embodiments, the anti-CD38 monoclonal antibody is daratumumab and/or the anti-SLAMF7 monoclonal antibody is elotuzumab. In some embodiments of the method of treating, the method further comprises administering to the subject a daily dose of one or more chemotherapeutic agents for three consecutive days, wherein the duration between the last daily dose administration of the one or more chemotherapeutic agents and a first weekly dose of the adoptive cell therapy product is about 40-84 hours. In some embodiments, the one or more chemotherapeutic agents comprise cyclophosphamide (CY) and fludarabine (FLU), and optionally wherein the daily dose of CY is at about 500 mg/m² and the daily dose of FLU is at about 30 mg/m².

In various embodiments of the method of treating, the method: (a) does not require CY/FLU-based lympho-conditioning; or (b) requires a minimal need of CY/FLU-based lympho-conditioning; and wherein the step (ii) of the method comprises administering to the subject weekly doses of an effective amount of the anti-CD38 monoclonal antibody. In some embodiments, the method does not require IL2 cytokine support to the subject during the course of treatment. In some embodiments, the engineered NK lineage cell is derived from an engineered induced pluripotent stem cell (iPSC) comprising CD38 knockout, a polynucleotide encoding an exogenous CD16, and a polynucleotide encoding IL15RF. In some embodiments, the effective amount of the adoptive cell therapy product in a dose is about 5×10⁷ cells/dose to 3×10⁹ cells/dose. In some embodiments, the effective amount of the adoptive cell therapy product in each dose is about 1×10⁸, 3×10⁸, 10×10⁸, or about 1.5×10⁹ cells. In some embodiments, (i) the daratumumab is in an effective amount of about 15 mg/kg to about 17 mg/kg; and/or (ii) the elotuzumab is in an effective amount of about 9 mg/kg to about 11 mg/kg. In some embodiments, (i) the effective amount of daratumumab is about 16 mg/kg; and (ii) the effective amount of elotuzumab is about 10 mg/kg.

In various embodiments of the method of treating, the method further comprises assessing disease response after a first cycle of administration of the adoptive cell therapy product. In some embodiments, assessing disease response comprises assaying a bone marrow biopsy, a peripheral blood sample, and a urine sample from the subject for complete response or partial response based on criteria comprising leukemic blast count, absolute neutrophil count and/or platelet count, wherein: (i) the complete response comprises: (a) a negative immunofixation on serum from the subject, as compared to serum immunofixation prior to the course of treatment; (b) a negative immunofixation on urine from the subject, as compared to urine immunofixation prior to the course of treatment; (c) elimination of soft tissue plasmacytomas, as compared to plasmacytomas prior to the course of treatment; and/or (d) <5% plasma cells in bone marrow, as compared to plasma cells in bone marrow prior to the course of treatment; and/or (ii) the partial response comprises: (a) ≥25% but ≤49% reduction of serum M-protein level, as compared to serum M-protein prior to the course of treatment; and/or (b) reduction in 24-h urine M-protein level by about 50-89%, as compared to urine M-protein prior to the course of treatment. In some embodiments, the administering of the adoptive cell therapy product is (i) via intravenous infusion, and/or (ii) at a site of an outpatient setting; and/or wherein each dose of the adoptive cell therapy product is cryopreserved, and then thawed prior to administering. In various embodiments of the method of treating, the BCMA directed CAR comprises: (i) a variable heavy chain (VH) and a variable light chain (VL), wherein: (a) said VH comprises: a heavy chain complementary determining region 1 (H-CDR1) with at least 80% sequence identity (e.g., 90% or 100% identity) to SEQ ID NO: 8 (GFTFSRYW), a heavy chain complementary determining region 2 (H-CDR2) with at least 80% sequence identity (e.g., 90% or 100% identity) to SEQ ID NO: 9 (INPSSSTI), and a heavy chain complementary determining region 3 (H-CDR3) with at least 80% sequence identity (e.g., 90% or 100% identity) to SEQ ID NO: 10 (ASLYYDYGDAYDY); and (b) a variable light chain (VL), said VL comprising: a light chain complementary determining region 1 (L-CDR1) with at least 80% sequence identity (e.g., 90% or 100% identity) to SEQ ID NO: 11 (QSVESN), a light chain complementary determining region 2 (L-CDR2) with at least 80% sequence identity (e.g., 90% or 100% identity) to SEQ ID NO: 12 (SAS), and a light chain complementary determining region 3 (L-CDR3) with at least 80% sequence identity (e.g., 90% or 100% identity) to SEQ ID NO: 13 (QQYNNYPLT); or (ii) an amino acid sequence that is of at least about 99%, 98%, 96%, 95%, 90%, 85%, or 80% identity to SEQ ID NO: 7. In some embodiments, the BCMA directed CAR comprises the amino acid sequence of SEQ ID NO: 7.

In another aspect, the present invention provides a method of a multi-dose targeted adoptive cell therapy in a subject in need thereof comprising: (i) weekly administration to the subject of an effective amount of the targeted adoptive cell therapy product for a course of treatment of about three weeks, wherein the product comprises: an engineered immune cell expressing CD16, IL15RF, and a BCMA directed CAR, and wherein the engineered immune cell is CD38 negative; and (ii) detecting and comparing one or more of the following at different given time points following administration of a first dose of the adoptive cell therapy: (a) the presence of the engineered immune cell in bone marrow of the subject; (b) the presence of the engineered immune cell in a tumor of the subject; (c) protein markers of disease in serum of the subject; (d) cytokines in a peripheral blood sample from the subject; and (e) circulating tumor DNA in a peripheral blood sample from the subject, wherein any of (a)-(e) is useful for assessing tumor burden, tumor immunobiology, and/or tumor therapy response, thereby determining efficacy of the multi-dose targeted adoptive cell therapy. In some embodiments, the subject has multiple myeloma (MM). In some embodiments, the effective amount of the adoptive cell therapy product is about 5×10⁷ cells/dose to 1.5×10⁹ cells/dose.

In various embodiments of the method of a multi-dose targeted adoptive cell therapy, the method further comprises weekly administration to the subject of an effective amount of an anti-CD38 monoclonal antibody, or an anti-SLAMF7 monoclonal antibody, wherein a first dose of the anti-CD38 monoclonal antibody or an anti-SLAMF7 monoclonal antibody precedes step (i) by about 10 days. In some embodiments, the anti-CD38 monoclonal antibody is daratumumab and/or the anti-SLAMF7 monoclonal antibody is elotuzumab, and/or the anti-CD20 monoclonal antibody is rituximab. In some embodiments, the BCMA directed CAR comprises: (i) a variable heavy chain (VH) and a variable light chain (VL), wherein: (a) said VH comprises: a heavy chain complementary determining region 1 (H-CDR1) with at least 80% sequence identity (e.g., 90% or 100% identity) to SEQ ID NO: 8 (GFTFSRYW), a heavy chain complementary determining region 2 (H-CDR2) with at least 80% sequence identity (e.g., 90% or 100% identity) to SEQ ID NO: 9 (INPSSSTI), and a heavy chain complementary determining region 3 (H-CDR3) with at least 80% sequence identity (e.g., 90% or 100% identity) to SEQ ID NO: 10 (ASLYYDYGDAYDY); and (b) a variable light chain (VL), said VL comprising: a light chain complementary determining region 1 (L-CDR1) with at least 80% sequence identity (e.g., 90% or 100% identity) to SEQ ID NO: 11 (QSVESN), a light chain complementary determining region 2 (L-CDR2) with at least 80% sequence identity (e.g., 90% or 100% identity) to SEQ ID NO: 12 (SAS), and a light chain complementary determining region 3 (L-CDR3) with at least 80% sequence identity (e.g., 90% or 100% identity) to SEQ ID NO: 13 (QQYNNYPLT); or (ii) an amino acid sequence that is of at least about 99%, 98%, 96%, 95%, 90%, 85%, or 80% identity to SEQ ID NO: 7. In some embodiments, the BCMA directed CAR comprises the amino acid sequence of SEQ ID NO: 7.

In another aspect, the present invention provides a method of retreatment following one or more earlier line(s) of treatment of MM according to the methods provided herein, wherein the one or more early lines of therapy for MM comprise: (a) a chemotherapy, an immunochemotherapy, hematopoietic stem cell transplantation, chimeric antigen receptor (CAR) T-cell therapy, or any combination thereof; (b) a proteasome inhibitor, an anti-CD38 antibody, an anti-SLAMF7 antibody, an immunomodulatory drug, a stem-cell transplantation (SCT), or any combination thereof, (c) bortezomib, carfilzomib, ixazomib, daratumumab, isatuximab, elotuzumab, thalidomide, lenalidomide, pomalidomide, or any combination thereof, and/or (d) idecabtagene vicleucel (bb2121, ide-cel), a BCMA-directed autologous CAR T-cell therapy.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIG. 1 is a graphic representation showing an exemplary iNK cell for use in an immunotherapy described herein.

### DETAILED DESCRIPTION OF THE INVENTION

Genomic modification of iPSCs (induced pluripotent stem cells) includes polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cell), T cell progenitor cells, NK cell progenitor cells, T cells, NKT cells, NK cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, refers to stem cells that are produced in vitro, using reprogramming factor and/or small molecule chemical driven methods, from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) the ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain,” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3⁺ cells. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”.

“CD4⁺ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 or CD16 and the absence of the T cell receptor (CD3). An NK cell can be any NK cell, such as a cultured NK cell, e.g., a primary NK cell, or an NK cell from a cultured or expanded NK cell or a cell-line NK cell, e.g., NK-92, or an NK cell obtained from a mammal that is healthy or with a disease condition. As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”.

As used herein, the term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified with the expression of at least one or more of the following markers, TCR Va24-Ja18, Vb11, CD1d, CD3, CD4, CD8, aGalCer, CD161 and CD56.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained from separating the desired cells, or populations thereof, from other substances or cells in the environment, or from removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. “Polynucleotide” also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” refers to each separate polypeptide chain of a protein complex, where each separate polypeptide chain can form a stable folded structure by itself. Many protein molecules are composed of more than one subunit, where the amino acid sequences can either be identical for each subunit, or similar, or completely different. For example, CD3 complex is composed of CD3α, CD3ε, CD3δ, CD3γ, and CD3ζ subunits, which form the CD3ε/CD3γ, CD3ε/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK cells or derivative T cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing. In the aspect of a source cell obtained from a specifically selected donor, disease or treatment context, the genetic imprint contributing to preferential therapeutic attributes may include any context specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, a synthetic cell possesses one or more non-native cell functions when compared to its closest counterpart primary cell.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy.

As used herein, the term “engager” refers to a molecule, e.g., a fusion polypeptide, which is capable of forming a link between an immune cell, e.g., a T cell, a NK cell, a NKT cell, a B cell, a macrophage, a neutrophil, and a tumor cell; and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, an NK cell, an NKT cell, a B cell, a macrophage, a neutrophil. In some embodiments, the surface triggering receptor facilitates bi- or multi-specific antibody engagement between the effector cells and specific target cell e.g., a tumor cell, independent of the effector cell's natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers having the same epitope recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some case, and to kill two or more types of tumors in some other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B-cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. “Pharmaceutically active proteins” include an entire protein or peptide or pharmaceutically active fragments thereof. The term also includes pharmaceutically active analogs of the protein or peptide or analogs of fragments of the protein or peptide. The term “pharmaceutically active protein” also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, the cellular signal transduction. “Signal transduction” refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes, but is not limited to, (i) antigen specificity as it relates to a unique chimeric antigen receptor (CAR) or T cell receptor (TCR), (ii) engager specificity as it relates to monoclonal antibodies or bi-specific engagers, (iii) targeting of transformed cells, (iv) targeting of cancer stem cells, and (v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogenic lymphocytes, whether the immune cells are isolated from a human donor or effector cells obtained from in vitro differentiation of a pluripotent cell; whether they are genetically modified or not; or whether they are primary donor cells or cells that have been passaged, expanded, or immortalized, ex vivo, after isolation from a donor.

As used herein, “lymphodepletion” and “lympho-conditioning” are used interchangeably to refer to the destruction of lymphocytes and T cells, typically prior to immunotherapy. The purpose of lympho-conditioning prior to the administration of an adoptive cell therapy is to promote homeostatic proliferation of effector cells as well as to eliminate regulatory immune cells and other competing elements of the immune system that compete for homeostatic cytokines. Thus, lympho-conditioning is typically accomplished by administering one or more chemotherapeutic agents to the subject prior to a first dose of the adoptive cell therapy. In various embodiments, lympho-conditioning precedes the first dose of the adoptive cell therapy by a few hours to a few days. Exemplary chemotherapeutic agents useful for lympho-conditioning include, but are not limited to, cyclophosphamide (CY), fludarabine (FLU), and those described below. However, a sufficient lymphodepletion through anti-CD38 mAb could provide an alternative conditioning process for the present iNK cell therapy, without or with minimal need of a CY/FLU-based lympho-conditioning procedure, as further described herein.

As used herein, “homing” or “trafficking” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of a cell to a target site.

As used herein, the term “outpatient” refers to a patient who is not hospitalized overnight, but who visits a hospital, clinic, or associated facility for diagnosis or treatment. Thus, an “outpatient setting,” as compared to an “inpatient setting” refers to an environment for providing ambulatory care or outpatient care to a patient where hospitalization for one or more days/nights is not required while receiving treatment or diagnosis, thereby reducing overall discomfort to the patient receiving treatment and/or diagnosis, while reducing overall cost for such treatment and/or diagnosis with relative ease in management and coordination. Additionally, an outpatient setting is more readily accessible to a larger population of patients and increases patient availability and patient compliance with treatment protocol during a trial or course of treatment.

As used herein, “induction therapy,” also called “first-line therapy,” “primary therapy,” or “primary treatment,” refers to a first treatment given to a patient for a particular disease. It is often part of a standard set of treatments, such as surgery followed by chemotherapy and radiation. Thus, an “induction attempt” or “attempt of induction therapy” refers to an initial attempt at treating a particular disease using known and/or conventional therapeutic approaches for the particular disease.

A “therapeutically sufficient amount,” as used herein, includes within its meaning a non-toxic but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a “therapeutically sufficient amount” is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate the lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation state because of the inconsistent exposure of the cells in the three-dimensional structure to differentiation cues from the environment. In addition, EBs are laborious to create and maintain. Moreover, cell differentiation through EB is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation for differentiation initiation. Because monolayer culturing does not mimic embryo development such as is the case with EB formation, differentiation towards specific lineages are deemed as minimal as compared to all three germ layer differentiation in EB formation.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level—successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, addition, or alteration of a cell function/characteristics via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, or HLA class II deficient, or both, refers to cells that either lack, or no longer maintain, or have a reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related, but not limited to improved differentiation potential, antigen targeting, antigen presentation, antibody recognition, persistence, immune evasion, resistance to suppression, proliferation, costimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 41BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” is used herein in the broadest sense and refers generally to an immune-response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcR). The classes of FcRs are also distinguished by the cells that express them (macrophages, granulocytes, natural killer cells, T and B cells) and the signaling properties of each receptor. Fc-gamma receptors (FcγR) includes several members, FcγRI (CD64), FcγRIIA (CD32), FcγRIIB (CD32), FcγRIIIA (CD16a), FcγRIIIB (CD16b), which differ in their antibody affinities due to their different molecular structures.

“Chimeric Receptor” is a general term used to describe an engineered, artificial, or a hybrid receptor protein molecule that is made to comprise two or more portions of amino acid sequences that are originated from at least two different proteins. Chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), as well as fusions of two or more receptors.

“Chimeric Fc Receptor,” abbreviated as “CFcR,” is a term used to describe engineered Fc receptors having their native transmembrane and/or intracellular signaling domains modified, or replaced with non-native transmembrane and/or intracellular signaling domains. In some embodiments of the chimeric Fc receptor, in addition to having one of, or both, transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR), which contains antigen binding domain to target antigen, the chimeric Fc receptor binds to an Fc fragment, or the Fc region of an antibody, or the Fc region comprised in an engager or a binding molecule and activating the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16 based CFcR, the extracellular domain of CD64 or the high-affinity variants of CD16 (F176V, for example) can be incorporated. In some embodiments of the CFcR where high affinity CD16 extracellular domain is involved, the proteolytic cleavage site comprising a serine at position 197 is eliminated or is replaced such at the extracellular domain of the receptor is non-cleavable, i.e., not subject to shedding, thereby obtaining a hnCD16-based CFcR.

CD16, a FcγR receptor, has been identified to have two isoforms: Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16 (abbreviated as hnCD16),” as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

As used herein, “FT576” refers to an iPSC-derived, off the-shelf, BCMA-directed chimeric antigen receptor (CAR) natural killer (NK) cell therapy having four engineered modalities: (1) high-affinity non-cleavable CD16 Fc receptor for augmented antibody-dependent cellular cytotoxicity (ADCC); (2) IL-15/IL-15 receptor fusion (3) CD38 knockout; and (4) a BCMA-directed CAR to target clonal plasma cells.

I. Cells and Compositions Useful for Adoptive Cell Therapies with Enhanced Properties

Provided herein is a strategy to systematically engineer the regulatory circuitry of a clonal iPSC without impacting the differentiation potency and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic editing still have the capacity to enter cell development, and/or to mature and generate functional differentiated cells while retaining modulated activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and need for reconfiguration of differentiation protocols enabling phenotypic and/or functional change in the cell. The present application has shown that the one or more selected genomic modifications as provided herein does not negatively impact iPSC differentiation potency, and the functional effector cells derived from the engineered iPSC have enhanced and/or acquired therapeutic properties attributable to the individual or combined genomic modifications retained in the effector cells following the iPSC differentiation.

### 1. CD38 Knockout

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include phagocytosis (ADCP) and/or complement-dependent cytotoxicity (CDC).

Other than being highly expressed on malignant cells, CD38 is also expressed on plasma cells as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the process of cell adhesion which process is calcium dependent. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38-specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114).

Without being limited by theories, the present application provides a strategy to leverage the full potential of CD38-targeted cancer treatment by knocking out CD38 in the effector cell, thereby overcoming CD38-specific antibody and/or CD38 antigen binding domain-induced effector cell depletion or reduction through fratricide. In addition, since CD38 is upregulated on activated lymphocytes such as T or B cells, by suppressing activation of these recipient lymphocytes using a CD38-specific antibody, such as daratumumab, in the recipient of allogeneic effector cells, host allorejection against these effector cells would be reduced and/or prevented, thereby increasing effector cell survival and persistency. As such, a CD38-specific antibody, a secreted CD38-specific engager or a CD38-CAR (chimeric antigen receptor) against activation of recipient T, Treg, NK, and/or B cells can be used as a replacement for lymphodepletion using chemotherapy such as Cy/Flu (cyclophosphamide/fludarabine) prior to adoptive cell transferring. In addition, when targeting CD38⁺ T and pbNK cells using hnCD16a⁺/CD38⁻ effector cells in the presence of anti-CD38 antibodies or CD38 inhibitors, the depletion of CD38⁺ alloreactive cells increases the NAD⁺ (nicotinamide adenine dinucleotide, a substrate of CD38) availability and decreases NAD⁺ consumption related cell death, which, among other advantages, boosts effector cell responses in an immunosuppressive tumor microenvironment and supports cell rejuvenation in aging, degenerative or inflammatory diseases.

Thus, strategies according to some embodiments include generating a CD38 knockout iPSC line and obtaining CD38 negative (CD38^{-/-}) derivative effector cells through directed differentiation of the engineered iPSC line. In one embodiment as provided herein, the CD38 knockout in an iPSC line is a bi-allelic knockout. As disclosed herein, the provided CD38^{-/-} iPSC line is capable of directed differentiation to produce functional derivative hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages. In some embodiments, when an anti-CD38 antibody is used to induce ADCC or an anti-CD38 CAR is used for targeted cell killing, the CD38^{-/-} iPSC and/or its derivative effector cells thereof are not eliminated by the anti-CD38 antibody or the anti-CD38 CAR, thereby increasing the persistence and/or survival of the iPSC and its derivative effector cell in the presence of, and/or after exposure to, such therapeutic agents. In some embodiments, the effector cell has increased persistence and/or survival in vivo in the presence of, and/or after exposure to, such therapeutic agents. In some embodiments, the CD38^{-/-} effector cells are NK cells derived from iPSCs. In some embodiments, the CD38^{-/-} iPSC and derivative cells comprise one or more additional genomic editing as described herein, including but not limited to, CD16 expression, and cytokine/cytokine receptor expression, and optionally, additional modalities as provided.

### 2. CD16 Knock-in

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” “high affinity non-cleavable CD16,” or “hnCD16,” as used herein, refers to various CD16 variants. The wildtype CD16 has low affinity and is subject to ectodomain shedding, a proteolytic cleavage process that regulates the cells surface density of various cell surface molecules on leukocytes upon NK cell activation. F176V (also called F158V in some publications) is an exemplary CD16 polymorphic variant having high affinity; whereas S197P variant is an example of genetically engineered non-cleavable version of CD16. An engineered CD16 variant comprising both F176V and S197P has high affinity and is non-cleavable, which was described in greater detail in WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

Unlike the endogenous CD16 receptor expressed by primary NK cells which gets cleaved from the cellular surface following NK cell activation, the various non-cleavable versions of CD16 in derivative NK avoids CD16 shedding and maintains constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, in some embodiments, the hnCD16 may comprise F176V and S197P, or may comprise a full or partial ectodomain originated from CD64, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed below.

Thus, in some embodiments, the derived NK cells are used in a combination therapy with an antibody. In some embodiments, the antibody in the combination therapy or preloaded with the derived effector cells specifically targets CD38. In some embodiments, the antibody in the combination therapy or preloaded with the derived effector cells specifically targets an antigen different from CD38. In some embodiments, the anti-CD38 antibody is daratumumab.

Unlike primary NK cells, mature T cells from a primary source (i.e., natural/native sources such as peripheral blood, umbilical cord blood, or other donor tissues) do not express CD16. It was unexpected that an iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR (chimerical antigen receptor). When said derivative T lineage cell comprises acquired ADCC through exogenous CD16, including functional variants and CD16-based CFcR, expression, and when an antibody targets a different tumor antigen from the one targeted by the CAR, the antibody can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, a hnCD16 comprises a sequence having identity of at least 50%, 55%, 60%, 65%, 70%, 75%, 80%, 85%, 90%, 95%, 99%, 100%, or any percentage in-between, when compared to any of the exemplary sequences, SEQ ID NOs: 1, 2 and 3, each comprises at least a portion of CD64 ectodomain. As used herein and throughout the application, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm recognized in the art.

SEQ ID NO: 1
MWFLTTLLLWVPVDGQVDTTKAVITLQPPWVSVFQEETVTLHCEVLHLP
GSSSTQWFLNGTATQTSTPSYRITSASVNDSGEYRCQRGLSGRSDPIQL
EIHRGWLLLQVSSRVFTEGEPLALRCHAWKDKLVYNVLYYRNGKAFKFF
HWNSNLTILKTNISHNGTYHCSGMGKHRYTSAGISVTVKELFPAPVLNA
SVTSPLLEGNLVTLSCETKLLLQRPGLQLYFSFYMGSKTLRGRNTSSEY
QILTARREDSGLYWCEAATEDGNVLKRSPELELQVLGLQLPTPVWFHYQ
VSFCLVMVLLFAVDTGLYFSVKTNIRSSTRDWKDHKFKWRKDPQDK
(340 a.a. CD64 domain-based construction;
CD16TM; CD16ICD)
SEQ ID NO: 2
MWFLTTLLLWVPVDGQVDTTKAVITLQPPWVSVFQEETVTLHCEVLHLP
GSSSTQWFLNGTATQTSTPSYRITSASVNDSGEYRCQRGLSGRSDPIQL
EIHRGWLLLQVSSRVFTEGEPLALRCHAWKDKLVYNVLYYRNGKAFKFF
HWNSNLTILKTNISHNGTYHCSGMGKHRYTSAGISVTVKELFPAPVLNA
SVTSPLLEGNLVTLSCETKLLLQRPGLQLYFSFYMGSKTLRGRNTSSEY
QILTARREDSGLYWCEAATEDGNVLKRSPELELQVLGLFFPPGYQVSFC
LVMVLLFAVDTGLYFSVKTNIRSSTRDWKDHKFKWRKDPQDK
(336 a.a. CD64 exon-based construction;
CD16TM; CD16ICD)
SEQ ID NO: 3
MWFLTTLLLWVPVDGQVDTTKAVITLQPPWVSVFQEETVTLHCEVLHLP
GSSSTQWFLNGTATQTSTPSYRITSASVNDSGEYRCQRGLSGRSDPIQL
EIHRGWLLLQVSSRVFTEGEPLALRCHAWKDKLVYNVLYYRNGKAFKFF
HWNSNLTILKTNISHNGTYHCSGMGKHRYTSAGISVTVKELFPAPVLNA
SVTSPLLEGNLVTLSCETKLLLQRPGLQLYFSFYMGSKTLRGRNTSSEY
QILTARREDSGLYWCEAATEDGNVLKRSPELELQVLGFFPPGYQVSFCL
VMVLLFAVDTGLYFSVKTNIRSSTRDWKDHKFKWRKDPQDK
(335 a.a. CD64 exon-based construction; CD16TM;
CD16ICD)

Accordingly, provided herein are iPSCs genetically engineered to comprise, among other editing as contemplated and described herein, an exogenous CD16 that is a high-affinity non-cleavable CD16 receptor (hnCD16), wherein the genetically engineered iPSCs are capable of differentiating into effector cells comprising the hnCD16 introduced to the iPSCs. In some embodiments, the derived effector cells comprising hnCD16 are NK cells. The exogenous hnCD16 expressed in iPSC or derivative cells thereof has high affinity in binding to not only ADCC antibodies or fragments thereof, but also to bi-, tri-, or multi-specific engagers or binders that recognize the CD16 or CD64 extracellular binding domains of said hnCD16. As such, the present application provides a derivative effector cell or a cell population thereof, preloaded with one or more pre-selected ADCC antibody through high-affinity binding with the extracellular domain of the hnCD16 expressed on the derivative effector cell, in an amount sufficient for therapeutic use in a treatment of a condition, a disease, or an infection as further detailed below, wherein said hnCD16 comprises an extracellular binding domain of CD64, or of CD16 having F176V and S197P.

In some other embodiments, the native CD16 transmembrane- and/or the intracellular-domain of a hnCD16 is further modified or replaced, such that a chimeric Fc receptor (CFcR) is produced to comprise a non-native transmembrane domain, a non-native stimulatory domain and/or a non-native signaling domain. The term “non-native” used herein means that the transmembrane, stimulatory or signaling domain are derived from a different receptor other than the receptor which provides the extracellular domain. In the illustration here, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cel receptor polypeptide. In some embodiments, the exogenous CD16-based CFcR comprises a non-native stimulatory/inhibitory domain derived from CD27, CD28, 4-1BB, OX40, ICOS, PD-1, LAG-3, 2B4, BTLA, DAP10, DAP12, CTLA-4, or NKG2D polypeptide. In some embodiments, the exogenous CD16-based CFcR comprises a non-native signaling domain derived from CD3ζ, 2B4, DAP10, DAP12, DNAM1, CD137 (41BB), IL21, IL7, IL12, IL15, NKp30, NKp44, NKp46, NKG2C, or NKG2D polypeptide. In some embodiments, the CD16-based Fc receptor comprises a transmembrane domain and a signaling domain both derived from one of IL7, IL12, IL15, NKp30, NK p44, NKp46, NKG2C, and NKG2D polypeptide. One particular exemplary embodiment of the CD16-based chimeric Fc receptor comprises a transmembrane domain of NKG2D, a stimulatory domain of 2B4, and a signaling domain of CD3ζ; wherein the extracellular domain of the CFcR is derived from a full length or partial sequence of the extracellular domain of CD64 or CD16, wherein the extracellular domain of CD16 comprises F176V and S197P. Another exemplary embodiment of the CD16-based chimeric Fc receptor comprises a transmembrane domain and a signaling domain of CD3ζ; wherein the extracellular domain of the hnCD16 is derived from a full length or partial sequence of the extracellular domain of CD64 or CD16, wherein the extracellular domain of CD16 comprises F176V and S197P.

The various embodiments of CD16-based chimeric Fc receptor as described above are capable of binding, with high affinity, to the Fc region of an antibody or fragment thereof, or to the Fc region of a bi-, tri-, or multi-specific engager or binder. Upon binding, the stimulatory and/or signaling domains of the chimeric receptor enable the activation and cytokine secretion of the effector cells, and the killing of the tumor cells targeted by the antibody, or said bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into a close proximity, which also contributes to the enhanced killing of the tumor cells. Exemplary tumor antigens for bi-, tri-, multi-specific engagers or binders include, but are not limited to, B7H3, BCMA, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EPCAM, and CD16 (or CD64)-IL15-CD33.

Accordingly, in some embodiments, the present invention provides iPSCs and derivative cells therefrom comprising exogenous CD16 and CD38 knockout (CD38^{-/-}). In some embodiments, the derivative cells comprise derived NK cells (iNK cells) comprising exogenous CD16 and CD38 knockout. In some embodiments the iNK cells comprising exogenous CD16 and CD38 knockout are preloaded with anti-CD38 antibody. In some embodiments, the preloaded anti-CD38 antibody is daratumumab. In some embodiments the iNK cells comprising exogenous CD16 and CD38 knockout further comprise one or more additional genomic editing as described herein, including but not limited to, cytokine/cytokine receptor expression, and optionally, additional modalities as provided herein.

### 3. Exogenously Introduced Cytokine Signaling Complex

By avoiding systemic high-dose administration of clinically relevant cytokines, the risk of dose-limiting toxicities due to such a practice is reduced while cytokine mediated cell autonomy being established. To achieve lymphocyte autonomy without the need to additionally administer soluble cytokines, a cytokine signaling complex comprising a partial or full length peptide of one or more of IL2, IL4, IL6, IL7, IL9, IL10, IL11, IL12, IL15, IL18, IL21, and/or their respective receptor may be introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling (signaling complex) are expressed on the cell surface. In some embodiments, the cytokine signaling is constitutively activated. In some embodiments, the activation of the cytokine signaling is inducible. In some embodiments, the activation of the cytokine signaling is transient and/or temporal.

Various construct designs for introducing a protein complex for signaling of cytokines including, but not limited to, IL2, IL4, IL6, IL7, IL9, IL10, IL11, IL12, IL15, IL18 and IL21, into the cell are provided herein. In embodiments where the signaling complex is for IL15, the transmembrane (TM) domain can be native to the IL15 receptor or may be modified or replaced with transmembrane domain of any other membrane bound proteins. In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with truncated intracellular domain is fused to IL15 at the C-terminus through a linker, mimicking trans-presentation of IL15, maintaining IL15 membrane-bound, and additionally eliminating cis-presentation and/or any other potential signal transduction pathways mediated by a normal IL15R through its intracellular domain.

In some embodiments, such a truncated construct comprises an amino acid sequence of at least 75%, 80%, 85%, 90%, 95% or 99% identity to SEQ ID NO: 4. In one embodiment of the truncated IL15/IL115Rα, the construct does not comprise the last 4 amino acids “KSRQ” of SEQ ID NO: 4, and comprises an amino acid sequence of at least 75%, 80%, 85%, 90%, 95% or 99% identity to SEQ ID NO: 5. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 4
MDWTWILFLVAAATRVHSGIHVFILGCFSAGLPKTEANWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTSSGGGSGGGGSGGGGSGGGGSGGGSLQITCPPPMSVEHADIWVKSY
SLYSRERYICNSGFKRKAGTSSLTECVLNKATNVAHWTTPSLKCIRDPA
LVHQRPAPPSTVTTAGVTPQPESLSPSGKEPAASSPSSNNTAATTAAIV
PGSQLMPSKSPSTGTTEISSHESSHGTPSQTTAKNWELTASASHQPPGV
YPQGHSDTTVAISTSTVLLCGLSAVSLLACYLKSRQ
(379 a.a .; signal and linker peptides are
underlined)
SEQ ID NO: 5
MDWTWILFLVAAATRVHSGIHVFILGCFSAGLPKTEANWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTSSGGGSGGGGSGGGGSGGGGSGGGSLQITCPPPMSVEHADIWVKSY
SLYSRERYICNSGFKRKAGTSSLTECVLNKATNVAHWTTPSLKCIRDPA
LVHQRPAPPSTVTTAGVTPQPESLSPSGKEPAASSPSSNNTAATTAAIV
PGSQLMPSKSPSTGTTEISSHESSHGTPSQTTAKNWELTASASHQPPGV
YPQGHSDTTVAISTSTVLLCGLSAVSLLACYL
(375 a.a .; signal and linker peptides are
underlined)

In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the trans-membrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of IL15 is retained. In some embodiments, the component comprising IL15 fused with Sushi domain comprises an amino acid sequence of at least 75%, 80%, 85%, 90%, 95% or 99% identity to SEQ ID NO: 6.

SEQ ID NO: 6
MDWTWILFLVAAATRVHSGIHVFILGCFSAGLPKTEANWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTSSGGGSGGGGSGGGGSGGGGSGGGSLQITCPPPMSVEHADIWVKSY
SLYSRERYICNSGFKRKAGTSSLTECVLNKATNVAHWTTPSLKCIR
(242 a.a .; signal and linker peptides are
underlined)

In other embodiments, a native or modified IL15Rβ is fused to IL15 at the C-terminus through a linker, enabling constitutive signaling and maintaining IL15 membrane-bound and trans-representation. In other embodiments, a native or modified common receptor γC is fused to IL15 at the C-terminus through a linker for constitutive signaling and membrane bound trans-presentation of the cytokine. The common receptor γC is also called the common gamma chain or CD132, which is also known as IL2 receptor subunit gamma or IL2RG. γC is a cytokine receptor sub-unit that is common to the receptor complexes for many interleukin receptors, including, but not limited to, IL2, IL4, IL7, IL9, IL15 and IL21 receptor. In other embodiments, engineered IL15Rβ that forms a homodimer in the absence of IL15 is useful for producing constitutive signaling of the cytokine.

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled in the art understands that the signal peptide and/or linker sequences may be substituted for another sequence without altering the activity of the functional peptide led by the signal peptide or linked by the linker.

As such, in various embodiments, the cytokine IL15 and/or its receptor, may be introduced to iPSC using one or more of the construct designs described above, and to its derivative cells upon iPSC differentiation. In addition to an induced pluripotent stem cell (iPSC), a clonal iPSC, a clonal iPS cell line, or iPSC-derived cells comprising at least one engineered modality as disclosed herein are provided. Also provided is a master cell bank comprising single cell sorted and expanded clonal engineered iPSCs having at least an exogenously introduced cytokine and/or cytokine receptor signaling as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

In some embodiments, the iPSC and derivative cells comprising CD38 knockout (CD38^{-/-}) and exogenous CD16 further comprise a cytokine signaling complex. In some embodiments, the derivative cells comprise derived NK cells (iNK cells) comprising CD38 knockout, exogenous CD16 and a cytokine signaling complex. In some embodiments the iNK cells comprising CD38 knockout, exogenous CD16 and a cytokine signaling complex further comprise one or more additional genomic editing as described herein.

### 4. HLA-I- and HLA-II-Deficiency

Multiple HLA class I and class II proteins must be matched for histocompatibility in allogeneic recipients to avoid allogeneic rejection problems. Provided herein is an iPSC cell line with optionally eliminated or substantially reduced expression of both HLA class I (“HLA-I”) and HLA class II (“HLA-II”) proteins. HLA class I deficiency can be achieved by functional deletion of any region of the HLA class I locus (chromosome 6p21), or deletion or reducing the expression level of HLA class-I associated genes including, not being limited to, beta-2 microglobulin (B2M) gene, TAP 1 gene, TAP 2 gene and Tapasin. For example, the B2M gene encodes a common subunit essential for cell surface expression of all HLA class I heterodimers. B2M negative cells are HLA-I deficient.

HLA class II deficiency can be achieved by functional deletion or reduction of HLA-II associated genes including, not being limited to, RFXANK, CIITA, RFX5 and RFXAP. CIITA is a transcriptional coactivator, functioning through activation of the transcription factor RFX5 required for class II protein expression. CIITA negative cells are HLA-II deficient. However, lacking HLA class I expression increases susceptibility to lysis by NK cells. As such, this application provides an iPSC and derivative cells therefrom comprising HLA-I and/or HLA-II deficiency, for example by lacking B2M and/or CIITA expression, wherein the obtained derivative effector cells enable allogeneic cell therapies by eliminating the need for MHC (major histocompatibility complex) matching, and avoiding recognition and killing by host (allogeneic) T cells.

Furthermore, a lack of HLA class I expression leads to lysis by host NK cells. Therefore, in addition to the above-discussed approach of CD38 conditioning to remove activated CD38-expressing host NK cells, to overcome this “missing self” response, HLA-G may be optionally knocked in to avoid NK cell recognition and killing of the HLA-I deficient effector cells derived from an engineered iPSC. In one embodiment, the provided HLA-I deficient iPSC and its derivative cells further comprise HLA-G knock-in

In some embodiments, the iPSC and derivative cells comprising CD38 knockout (CD38^{-/-}), exogenous CD16 and a cytokine signaling complex further comprise HLA-I and/or HLA-II deficiency, without adversely impacting the differentiation potential of the iPSC and function of the derived effector cells including derivative T and NK cells.

5. Genetically Engineered iPSC Line and Derivative Cells Provided Herein

In light of the above, the present application provides a CD38^{-/-} (also referred to as “CD38 negative” or CD38 knockout herein) iPSC, cell line cell, or a population thereof, and derived functional derivative cells comprising CD38 knockout obtained from differentiation of the CD38^{-/-} iPSC. In some embodiments, the functional derivative cells are hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T lineage cells, NKT lineage cells, NK lineage cells, B lineage cells, neutrophils, dendritic cells, and macrophages. In some embodiments, the functional derivative hematopoietic cells comprise effector cells having one or more functional features that are not present in a counterpart primary T, NK, NKT, and/or B cell.

Further provided herein is an iPSC comprising a CD38 knockout, and a polynucleotide encoding an exogenous CD16, wherein the iPSC is capable of directed differentiation to produce functional derivative hematopoietic cells. In some embodiments, when an anti-CD38 antibody is used to induce the CD16 mediated enhanced ADCC, the iPSC and/or its derivative effector cells can target the CD38 expressing (tumor) cells without causing effector cell elimination, i.e., reduction or depletion of CD38 expressing effector cells, thereby increasing the iPSC and its effector cell persistence and/or survival. In some embodiments, the effector cell has increased persistence and/or survival in vivo in the presence of anti-CD38 therapeutic agents, which may be an anti-CD38 antibody. In addition, since CD38 is upregulated on activated lymphocytes such as T or B cells, a CD38 specific antibody can be used for lymphodepletion thereby eliminating those activated lymphocytes, overcoming allorejection, and increasing survival and persistency of the CD38^{-/-} effector cells without fratricide in the recipient of the allogeneic effector cell therapy. In some embodiments, the effector cells comprise NK lineage cells. iPSC-derived NK lineage cells comprising CD38^{-/-} and exogenous CD16 have enhanced cytotoxicity and have reduced NK cell fratricide in the presence of anti-CD38 antibodies.

Additionally provided is an iPSC comprising a CD38 knockout, exogenous CD16 and a polynucleotide encoding at least one exogenous cytokine signaling complex (IL) to enable cytokine signaling contributing to cell survival, persistence and/or expansion, wherein the iPSC line is capable of hematopoietic differentiation to produce functional derivative effector cells having improved survival, persistency, expansion, and effector function. The exogenously introduced cytokine signaling(s) comprise the signaling of any one, or two, or more of IL2, IL4, IL6, IL7, IL9, IL10, IL11, IL12, IL15, IL18, and IL21. In some embodiments, the introduced cytokine signaling complex is expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through a retrovirus, Sendai virus, an adenovirus, an episome, mini-circle, or RNAs including mRNA. In some embodiments, the exogenous cytokine signaling complex comprised in the CD38^{-/-} iPSC or derivative cells thereof enables IL15 signaling. Said iPSC and derivative cells thereof comprising CD38^{-/-}, exogenous CD16, and IL of the above embodiments are capable of maintaining or improving cell growth, proliferation, expansion, and/or effector function autonomously without contacting additionally supplied soluble cytokines in vitro or in vivo, and can be used with an ADCC-capable antibody for targeted killing. When an anti-CD38 antibody is used with said CD38^{-/-} cell in a combination therapy, said cells have synergistically increased persistence, survival and effector function.

Also provided is an iPSC comprising a CD38 knockout, a B2M knockout and/or a CIITA knockout, and optionally, a polynucleotide encoding HLA-G, wherein the iPSC is capable of directed differentiation to produce functional derivative hematopoietic cells. Said CD38^{-/-} B2M^{-/-} CIITA^{-/-} iPSC and its derivative effector cells are both HLA-I and HLA-II deficient, and can be used with an anti-CD38 antibody to induce ADCC without causing effector cell elimination, thereby increasing the iPSC and its effector cell persistence and/or survival. In some embodiments, the effector cell has increased persistence and/or survival in vivo.

In view of the above, provided herein is an iPSC comprising a CD38 knockout, exogenous CD16 and exogenous cytokine signaling complex, and optionally B2M/CIITA knockout; wherein when B2M is knocked out, a polynucleotide encoding HLA-G is optionally introduced, and wherein the iPSC is capable of directed differentiation to produce functional derivative hematopoietic cells, wherein the derivative hematopoietic cells include, but are not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, macrophages, or a derivative effector cell having one or more functional features that are not present in a counterpart primary T, NK, NKT, and/or B cell.

6. Antibodies for immunotherapy

In some embodiments, in addition to the genomically engineered effector cells as provided herein, additional therapeutic agents comprising an antibody, or an antibody fragment that targets an antigen associated with a condition, a disease, or an indication may be used with these effector cells in a combinational therapy. In some embodiments, the antibody is used in combination with a population of the effector cells described herein by concurrent or consecutive administration to a subject. In other embodiments, such antibody or a fragment thereof may be expressed by the effector cells by genetically engineering an iPSC using an exogenous polynucleotide sequence encoding the antibody or fragment thereof, and directing differentiation of the engineered iPSC. In some embodiments, the effector cell additionally expresses an exogenous CD16 variant, wherein the cytotoxicity of the effector cell is enhanced by the antibody via ADCC. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 (rituximab, veltuzumab, ofatumumab, ublituximab, ocaratuzumab, obinutuzumab), anti-HER2 (trastuzumab, pertuzumab), anti-CD52 (alemtuzumab), anti-EGFR (cetuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab), and their humanized or Fc modified variants or fragments or their functional equivalents and biosimilars.

In some embodiments of the combination therapy comprising the derivative cells provided herein and at least one antibody, said antibody is not produced by, or in, the derivative cells and is additionally administered before, with, or after the administering of the derivative cells. In some embodiments, the antibody that is administered before, with, or after the administering of the derivative cells is an anti-CD38 monoclonal antibody or an anti-SLAMF7 monoclonal antibody.

### 7. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or deceasing activity of checkpoint molecules, thereby block inhibitory checkpoints, restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern.

The present application therefore provides, in some embodiments, a therapeutic approach to overcome CI resistance by including genomically-engineered functional derivative cells as provided in a combination therapy with CI. In one embodiment of the combination therapy, the derivative cells are NK cells. In addition to exhibiting direct antitumor capacity, the derivative NK cells provided herein have been shown to resist PDL1-PD1 mediated inhibition, and to have the ability to enhance T cell migration, to recruit T cells to the tumor microenvironment, and to augment T cell activation at the tumor site. Therefore, the tumor infiltration of T cell facilitated by the functionally potent genomically-engineered derivative NK cells indicate that said NK cells are capable of synergizing with T cell targeted immunotherapies, including the checkpoint inhibitors, to relieve local immunosuppression and to reduce tumor burden.

In one embodiment, the derived NK cell for checkpoint inhibitor combination therapy comprises a CD38 knockout, exogenous CD16, and an exogenous cytokine signaling complex, and optionally B2M/CIITA knockout, wherein when B2M is knocked out, a polynucleotide encoding HLA-G is optionally included. In some embodiments, the above derivative NK cell additionally comprises deletion or reduced expression in at least one of TAP1, TAP2, Tapasin, NLRC5, PD1, LAG3, TIM3, RFXANK, RFX5, RFXAP, and any gene in the chromosome 6p21 region; or introduced or increased expression in at least one of HLA-E, 41BBL, CD3, CD4, CD8, CD47, CD113, CD131, CD137, CD80, PDL1, A2AR, CAR, TCR, Fc receptor, an engager, and surface triggering receptor for coupling with bi-, multi-specific or universal engagers.

The above described derivative NK cell may be obtained from differentiating an iPSC clonal line cell comprising a CD38 knockout, exogenous CD16, and an exogenous cytokine signaling complex, and optionally B2M/CIITA knockout, wherein when B2M is knocked out, a polynucleotide encoding HLA-G is optionally introduced. In some embodiments, said iPSC clonal line cell further comprises deletion or reduced expression in at least one of TAP1, TAP2, Tapasin, NLRC5, PD1, LAG3, TIM3, RFXANK, RFX5, RFXAP, and any gene in the chromosome 6p21 region; or introduced or increased expression in at least one of HLA-E, 41BBL, CD3, CD4, CD8, CD47, CD113, CD131, CD137, CD80, PDL1, A2AR, CAR, TCR, Fc receptor, an engager, and surface triggering receptor for coupling with bi-, multi-specific or universal engagers.

Suitable checkpoint inhibitors for combination therapy with the derivative NK cells as provided herein include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2AR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

Some embodiments of the combination therapy with the provided derivative NK cells comprise at least one checkpoint inhibitor to target at least one checkpoint molecule. In some embodiments of the combination therapy comprising at least one checkpoint inhibitor, said checkpoint inhibitor is an antibody, or a humanized or Fc modified variant or fragment, or a functional equivalent or biosimilar thereof, and said checkpoint inhibitor is produced by the derivative cells by expressing an exogenous polynucleotide sequence encoding said antibody, or a fragment or variant thereof. In some embodiments, the exogenous polynucleotide sequence encoding the antibody, or a fragment or a variant thereof that inhibits a checkpoint is co-expressed with a chimeric antigen receptor (CAR), either in separate constructs or in a bi-cistronic construct comprising both CAR and the sequence encoding the antibody, or the fragment thereof. In some further embodiments, the sequence encoding the antibody or the fragment thereof can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR are in a single open reading frame (ORF). When the checkpoint inhibitor is delivered, expressed and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In some embodiments, the checkpoint inhibitor co-expressed with CAR inhibits at least one of the checkpoint molecules: PD-1, PDL-1, TIM-3, TIGIT, LAG-3, CTLA-4, 2B4, 4-1BB, 4-1BBL, A2AR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR.

In some embodiments, the checkpoint inhibitor co-expressed with CAR in a derivative cell described herein is selected from a group comprising atezolizumab, avelumab, durvalumab, tremelimumab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the derivative cells provided herein and at least one antibody inhibiting a checkpoint molecule, said antibody is not produced by, or in, the derivative cells and is additionally administered before, with, or after the administering of the derivative cells described herein. In some embodiments, the administering of one, two, three or more checkpoint inhibitors in a combination therapy with the provided derivative NK cells are simultaneous or sequential. In one embodiment of the combination treatment comprising derived NK cells described herein, the checkpoint inhibitor included in the treatment is one or more of atezolizumab, avelumab, durvalumab, tremelimumab, ipilimumab, IPH4102, IPH43, IPH33, lirimumab, monalizumab, nivolumab, pembrolizumab, and their humanized or Fc modified variants, fragments and their functional equivalents or biosimilars. In some embodiments of the combination treatment comprising derived NK cells described herein, the checkpoint inhibitor included in the treatment is atezolizumab, or its humanized or Fc modified variant, fragment and its functional equivalent or biosimilar. In some embodiments of the combination treatment comprising derived NK cells described herein, the checkpoint inhibitor included in the treatment is nivolumab, or its humanized or Fc modified variant, fragment or its functional equivalent or biosimilar. In some embodiments of the combination treatment comprising derived NK cells described herein, the checkpoint inhibitor included in the treatment is pembrolizumab, or its humanized or Fc modified variant, fragment or its functional equivalent or biosimilar.

II. Therapeutic Use of Derivative Immune Cells with Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed. In some embodiments, the iPSCs comprise one or more targeted genetic editing which are retainable in the iPSC-derived immune cells, wherein the genetically engineered iPSCs and derivative cells thereof are suitable for cell based adoptive therapies.

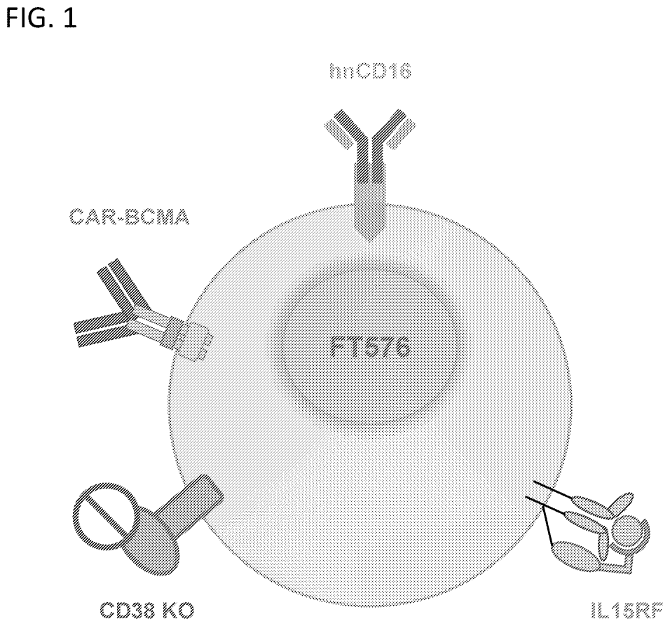

As shown in FIG. 1, the derivative immune cells comprised in the allogeneic natural killer (NK)-cell immunotherapy composition described herein, in accordance with some embodiments, are produced from a genomically engineered clonal master human-induced pluripotent stem cell (iPSC) line, and have an exogenous CD16 that is a high-affinity, non-cleavable CD16 receptor (hnCD16) (also referred to as “FT516” herein). In accordance with some other embodiments, the iPSC-derived NK cells comprised in the allogeneic cell immunotherapy composition have the following engineered elements: a) CD38 knockout; b) an exogenous CD16 that is a high-affinity, non-cleavable CD16 receptor (hnCD16); and c) a cytokine signaling complex comprising interleukin (IL)-15/IL-15 receptor alpha fusion protein (IL15RF) (also referred to as “FT538” herein). In the case of FT538, the clonal master cell bank (MCB) used for the production of the iNK cell therapy was generated by selecting and expanding a single well characterized iPSC clone in which a single IL15RF/hnCD16 expression cassette was inserted into the CD38 gene locus through non-viral-mediated targeted transgene integration.

The use of a clonal MCB as the starting material for current Good Manufacturing Practices (cGMP) production of the iNK cell therapy is intended to directly address many of the limitations associated with patient- and donor-specific cell therapies. Notably, many doses of the iNK cell therapy product can be uniformly produced in a single manufacturing campaign. These doses of drug product are homogeneous and are: (i) tested to assure compliance with a pre-defined quality specification, (ii) cryopreserved in an infusion medium, and (iii) stored to maintain a sustainable inventory. As such, the iNK cell therapy in the clinical setting has off-the-shelf availability for use in multi-dose regimens, which may prove critical for driving long-term durable responses in patients with progressing disease.

The engineered features of the iNK cell therapy are designed to result in increased activity against target tumor cells as monotherapy and when combined with monoclonal antibodies (mAbs) that can mediate antibody-dependent cellular cytotoxicity (ADCC). Functional attributes of the iNK cell therapy according to some embodiments include the following:
a) the iNK cell therapy product has superior effector function compared to patients' endogenous NK cells, which are typically diminished in number and function due to prior treatment regimens (e.g., chemotherapy) and tumor suppressive mechanisms. Thus, the iNK cell therapy mediates “innate cytotoxicity” that is potent and specific to transformed cells.
b) The CD38 gene KO in the iNK cell product prevents anti-CD38 antibody-mediated NK cell fratricide and consequently enhance ADCC when the iNK cell therapy is administered with concurrent anti-CD38 mAb therapy. In addition, NK cells with CD38 KO have more resistant to oxidative stress and exhibit enhanced effector function and persistence.
c) The iNK cells express an hnCD16 Fc receptor. The high-affinity CD16 variant arising from a naturally occurring 158V polymorphism has enhanced ADCC when combined with IgG mAbs including, but not limited to, rituximab, cetuximab, and trastuzumab. In addition, hnCD16 contains the genetic alteration (S179P) that prevents cleavage of CD16 by the metalloproteinase ADAM17, a mechanism in the regulation and attenuation of NK-cell activity by the tumor microenvironment.
d) The iNK cells express IL15RF, designed to provide an endogenous activation and proliferation signal, reducing the dependence on exogenous cytokine administration such as IL-2 and IL 15, both of which have been associated with significant toxicities that may limit clinical usage when incorporated into clinical studies of peripheral blood NK cells.

These features justify investigation of the iNK cells in a broad array of oncology indications including, but not limited to, the following: (i) as a monotherapy against, e.g., acute myelogenous leukemia (AML), where the iNK cells may provide greater clinical benefit than current allogeneic NK cell-based therapies; and (ii) in combination with approved and investigational tumor-targeting, ADCC-capable mAbs, including daratumumab and elotuzumab, which target CD38 and SLAMF7, respectively, and are approved for the treatment of patients with multiple myeloma (MM). Depending on the mAb used in the combination therapy, said iNK cells could also provide clinical benefit in treating solid cancer.

Previously, biodistribution and persistence studies of the iNK cells in immunodeficient NOD SCID-IL2rγ null (NSG) mice was evaluated following three IV injections at 3×10⁶ cells/mouse or 1.2×10⁷ cells/mouse administered 7 days apart on day of study 1, day of study 8, and day of study 15. The data demonstrated that the iNK cell product was detected in most tissues and that their persistence generally decreases over time to a level approaching or below the lower limit of detection by day 67 after infusion.

Thus, a variety of diseases may be ameliorated by introducing the immune cells of the invention to a subject suitable for adoptive cell therapy. In some embodiments, the iPSC derivative immune cells (e.g., iNK cells) as provided is for allogeneic adoptive cell therapies. Additionally, the present invention provides, in some embodiments, therapeutic use of the above therapeutic compositions by introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus. Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma (MM), and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's). Examples of viral infections include, but are not limited to, HIV- (human immunodeficiency virus), HSV- (herpes simplex virus), KSHV- (Kaposi's sarcoma-associated herpesvirus), RSV- (Respiratory Syncytial Virus), EBV- (Epstein-Barr virus), CMV- (cytomegalovirus), VZV (Varicella zoster virus), adenovirus-, a lentivirus-, a BK polyomavirus-associated disorders. In some embodiments, the hematological malignancies is refractory or relapsed. In particular embodiments, the patient receiving the adoptive cell therapy has acute myelogenous leukemia (AML), refractory or relapsed AML, secondary AML from myelodysplastic syndrome, multiple myeloma (MM), relapsed or refractory MM, non-Hodgkin's lymphoma (NHL), or relapsed or refractory NHL.

The treatment using the derived hematopoietic lineage cells of embodiments disclosed herein could be carried out upon symptom, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent or composition may be administered before, during or after the onset of a disease or an injury. The treatment of ongoing disease, where the treatment stabilizes or reduces the undesirable clinical symptoms of the patient, is also of particular interest. In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the derived hematopoietic lineage cells (e.g., iNK cells) of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

In some embodiments, the patient is monitored for any adverse events (AE) that may occur during the course of treatment. Exemplary adverse events include, but are not limited to, new malignancies, new or worsening neurologic disorders, new or worsening autoimmune or rheumatologic disorders, or new hematologic disorders. In some embodiments, the patient is monitored for cytokine release syndrome (CRS), neurotoxicity (ICAN) and/or GvHD before during and after the course of treatment.

The therapeutic composition comprising derived hematopoietic lineage cells as disclosed can be administered in a subject before, during, and/or after other treatments. As such the method of a combinational therapy can involve the administration or preparation of iPSC derived immune cells (e.g., iNK cells) before, during, and/or after the use of one or more additional therapeutic agents. As provided above, the one or more additional therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). Immunomodulatory drugs (IMiDs) such as thalidomide, lenalidomide, and pomalidomide stimulate both NK cells and T cells. As provided herein, IMiDs may be used with the iPSC derived therapeutic immune cells for cancer treatments. Additionally, or alternatively, the administration can be combined with other biologically active agents or modalities such as, but not limited to, an antineoplastic agent or a non-drug therapy, such as, surgery.

In some embodiments of a combinational cell therapy, the therapeutic combination comprises the iPSC derived hematopoietic lineage cells (e.g., iNK cells) provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC derived hematopoietic lineage cells to enhance their killing ability. In some embodiments, the antibody is a tumor-targeting, ADCC-capable monoclonal antibody (mAb). In some embodiments, the antibodies suitable for combinational treatment as an additional therapeutic agent to the administered iPSC derived hematopoietic lineage cells include, but are not limited to, anti-CD20 (e.g., rituximab, veltuzumab, ofatumumab, ublituximab, ocaratuzumab, obinutuzumab), anti-HER2 (e.g., trastuzumab, pertuzumab), anti-CD52 (e.g., alemtuzumab), anti-EGFR (e.g., cetuximab), anti-GD2 (e.g., dinutuximab), anti-PDL1 (e.g., avelumab), anti-CD38 (e.g., daratumumab, isatuximab, MOR202), anti-CD123 (e.g., 7G3, CSL362), anti-SLAMF7 (elotuzumab), and their humanized or Fc modified variants or fragments or their functional equivalents or biosimilars. In particular embodiments, the antibodies suitable for combinational treatment as an additional therapeutic agent to the administered iPSC derived hematopoietic lineage cells are daratumumab or elotuzumab.

In some embodiments, the additional therapeutic agent comprises one or more checkpoint inhibitors. Checkpoints are referred to cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells, as provided herein include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2aR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK cells as provided. In some embodiments, the derivative NK cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, avelumab, durvalumab, ipilimumab, IPH4102, IPH43, IPH33, lirimumab, monalizumab, nivolumab, pembrolizunab, and their derivatives or functional equivalents.

In some embodiments, other than the derivative effector cells as provided herein, a combination for therapeutic use comprises one or more additional therapeutic agents comprising a chemotherapeutic agent or a radioactive moiety. Chemotherapeutic agent refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide (CY), mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine (FLU), flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistframe.htm), both as updated from time to time. In particular embodiments, the chemotherapeutic agents suitable for combinational treatment as an additional therapeutic agent to the administered iPSC derived hematopoietic lineage cells are cyclophosphamide and fludarabine.

The combination therapies comprising the derivative effector cells and a monoclonal antibody, optionally in combination with one or more chemotherapeutic agents, are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et varioliformis acuta, CD30+ cutaneous T-cell lymphoma, Secondary cutaneous CD30+ large cell lymphoma, non-mycosis fungoides CD30 cutaneous large T-cell lymphoma, Pleomorphic T-cell lymphoma, Lennert lymphoma, subcutaneous T-cell lymphoma, angiocentric lymphoma, blastic NK-cell lymphoma, B-cell Lymphomas, Hodgkin lymphoma (HL), Head and neck tumor; Squamous cell carcinoma, rhabdomyocarcoma, Lewis lung carcinoma (LLC), non-small cell lung cancer, esophageal squamous cell carcinoma, esophageal adenocarcinoma, renal cell carcinoma (RCC), colorectal cancer (CRC), acute myelogenous leukemia (AML), multiple myeloma (MM), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma. In particular embodiments, the patient receiving the combination therapy has multiple myeloma (MM), or relapsed or refractory MM. In some embodiments, the patient receiving the combination therapy has AML, or relapsed or refractory AML. In some other embodiments, the patient receiving the combination therapy has NHL, or relapsed or refractory NHL.

The administration of the iPSC derived immune cells can be separated in time from the administration of any of the additional therapeutic agents by hours, days, or even weeks. In some embodiments, the administration of the iPSC derived immune cells can be preceded by administration of one or more chemotherapeutic agents alone or in combination with one or more antibodies. In particular embodiments, a subject may be administered a course of treatment comprising one or more chemotherapeutic agents daily for about 1-5 consecutive days, followed by a first or more doses or cycles of administration of the iPSC derived immune cells (e.g., iNK cells) of the invention. In some embodiments, the iNK cells are administered once per week for at least three weeks following daily administration of the one or more chemotherapeutic agents for three consecutive days. In one embodiment, the duration between the last dose of the one or more chemotherapeutic agents and the first dose of the iNK cells is about 40-84 hours.

In other embodiments of a combinational cell therapy, a subject may be administered a course of treatment comprising an anti-CD38 monoclonal antibody or an anti-SLAMF7 monoclonal antibody, followed by administration of one or more chemotherapeutic agents prior to being administered the iPSC derived immune cells (e.g., iNK cells) of the invention. In some embodiments, the anti-CD38 monoclonal antibody is daratumumab and the anti-SLAMF7 monoclonal antibody is elotuzumab. In some embodiments, the monoclonal antibody is administered weekly for 8 doses and two doses weekly ±1 day thereafter. In some other embodiments, the monoclonal antibody is administered every two weeks. In particular embodiments, the first dose of the weekly administered monoclonal antibody precedes administration of the one or more chemotherapeutic agents by about 4-6 days. In some embodiments, a subject may be administered a course of treatment comprising one or more chemotherapeutic agents daily for about 1-5 consecutive days, followed by weekly administration of the iPSC derived immune cells (e.g., iNK cells) of the invention. In some embodiments, the iNK cells are administered once per week for three weeks following daily administration of the one or more chemotherapeutic agents for three consecutive days. In one embodiment, the duration between the last dose of the one or more chemotherapeutic agents and the first dose of the iNK cells is about 40-84 hours or about 3 days.

Other than an isolated population of iPSC derived hematopoietic lineage cells included in the therapeutic compositions, the compositions suitable for administration to a patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of the present invention (see, e.g., Remington's Pharmaceutical Sciences, 17^{th} ed. 1985, the disclosure of which is hereby incorporated by reference in its entirety).

These pharmaceutically acceptable carriers and/or diluents can be present in amounts sufficient to maintain a pH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of the present invention. Such compositions are suitable for administration to human subjects. Generally speaking, any medium that supports the maintenance, growth, and/or health of the iPSC derived immune cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in the present invention and that there are many suitable media known and available to those in the art.

The isolated pluripotent stem cell derived hematopoietic lineage cells can have at least 50%, 60%, 70%, 80%, 90%, 95%, 98%, or 99% NK lineage cells. In some embodiments, the isolated pluripotent stem cell derived hematopoietic lineage cells have about 95% to about 100% NK cells. In some embodiments, the present invention provides therapeutic compositions having purified NK cells, such as a composition having an isolated population of about 95% NK cells to treat a subject in need of the cell therapy.

In one embodiment, the combinational cell therapy comprises an anti-CD38 therapeutic protein or peptide and a population of iNK cells derived from genomically engineered iPSCs, wherein the derived NK cells comprise CD38^{-/-} and express CD16 and IL15RF. In another embodiment, the combinational cell therapy comprises an anti-CD38 therapeutic protein or peptide and a population of iNK cells derived from genomically engineered iPSCs, wherein the derived NK cells comprise CD38^{-/-} and express CD16 and IL15RF. In some embodiments, the combinational cell therapy comprises daratumumab, isatuximab, or cyclophosphamide, and a population of NK derived from genomically engineered iPSCs, wherein the derived NK cells comprise CD38^{-/-}, hnCD16 and IL15RF.

As a person of ordinary skill in the art would understand, both autologous and allogeneic hematopoietic lineage cells derived from iPSC based on the methods and composition herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells.

In some embodiments, the number of derived NK lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 3×10⁸, at least 5×10⁹ cells, at least 1×10⁹ cells, at least 1.5×10⁹, or at least 5×10⁹ cells, per dose. In some embodiments, the number of derived NK lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to about 1.67×10⁶ cells/kg for a 60 kg patient.

In some embodiments, the number of derived hematopoietic lineage cells per dose being administered to a patient is calculated using dose fractionation, where the total number of cells to be administered is divided by the number of anticipated doses to be administered. In some embodiments, the number of cells per dose is the same for each dose (e.g., a ratio of 1:1:1, for three doses). In other embodiments, the ratio of the number of cells per dose is different for more than one dose (e.g., a ratio of 2:1:1, 1:1:2, or 1:2:1, for three doses).

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In some embodiments, the therapeutic use of the derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 5 days, every 7 days, every 10 days, every 15 days, every 20 days, every 25 days, every 30 days, every 35 days, every 40 days, every 45 days, or every 50 days, or any number of days in-between. In some embodiments, determination of the length and duration of the multi-dose treatment therapy is contingent on clinical observation of signs and/or symptoms (e.g., tumor size) of the disease or disorder being treated.

In some embodiments, the course of treatment may be repeated based on a review of clinical data demonstrating evidence of clinical benefit. In some embodiments, patients whose disease has an objective response to the iNK cell therapy, and later relapse or progress, may receive a second course of treatment.

The compositions comprising a population of derived hematopoietic lineage cells of the invention can be sterile, and can be suitable and ready for administration (i.e., can be administered without any further processing) to human patients. In some embodiments, the composition comprising a population of derived hematopoietic lineage cells of the invention is frozen in a bag or a vial ready to be thawed at the site of care under the condition as specified prior to administration. A cell based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more therapeutic agents. A therapeutic composition comprising a population of iPSC derived hematopoietic lineage cells as disclosed herein can be administered separately by intravenous, intraperitoneal, enteral, or tracheal administration methods or in combination with other suitable compounds to affect the desired treatment goals. In some embodiments, the adoptive cell therapy product is comprised in a composition that is administered via intravenous infusion; and/or at a site of an outpatient setting. In some embodiments, the adoptive cell therapy product is comprised in a cryopreserved container and thawed at the site of administration.

Some variation in dosage, frequency, and protocol will necessarily occur depending on the condition of the subject being treated. The person responsible for administration will, in any event, determine the appropriate dose, frequency and protocol for the individual subject within the ranges deemed safe and effective.

### EXAMPLES

The following examples are offered by way of illustration and not by way of limitation.

### Example 1—Materials and Methods

Adoptive Cell Therapy: The treatment regimens are based on administration of an adoptive cell therapy product that comprises an allogeneic iPSC-derived NK cell immunotherapy (i.e., iNK cell therapy), where the iPSC-derived NK (iNK) cells lack CD38 and express CD16 and IL15RF. Also disclosed here are the treatment regimens based on allogeneic iPSC-derived NK cells expressing CD16, without CD38 knockout or IL15RF expression. The iNK cells are suspended in infusion medium containing albumin (human) and DMSO and are provided in a cryopreserved bag and thawed at the site of administration. The iNK cell therapy is administered as an IV infusion via gravity with an in-line filter.

Prior to administration of the iNK cell therapy, subjects are pre-medicated with acetaminophen 650 mg orally and diphenhydramine 25 to 50 mg orally or IV before and 4 hours after administration. Corticosteroids are not used as pre-medication for the iNK cell therapy. Dosing is based on CD16 expression. Where 90%±10% of administered iNK cells express hnCD16, the starting dose for iNK cell monotherapy and in combination with the monoclonal antibodies is set to be about 1×10⁸ cells per dose. A dose level ranging from approximately 1×10⁸ to approximately 1×10¹⁰ cells is considered well tolerated as the starting dose of allogeneic NK cell therapies. Thus, the planned dosing levels (DL) of the NK cell therapy are: DL0: 5×10⁷ cells, DL1: 1×10⁸ cells, DL2: 3×10⁸ cells, DL3: 1×10⁹ cells, and DL4: 1.5×10⁹ cells, each of which is suitable for repeat administration with an expectation of tolerance and no dose-dependent toxicities.

Lympho-conditioning: The purpose of lympho-conditioning prior to the administration of iNK cell therapy is to promote homeostatic proliferation of iNK cells as well as to eliminate regulatory immune cells and other competing elements of the immune system that compete for homeostatic cytokines. CY is administered as an IV infusion at a dose of 500 mg/m² per institutional standard of care. CY dosing is calculated based on actual body weight (ABW). If ABW is >150% of the ideal body weight (IBW), then the dose is computed using adjusted body weight as follows: Adjusted body weight=IBW+0.5(ABW−IBW). FLU is administered as an IV infusion at a dose of 30 mg/m² per institutional standard. The duration between the last dose of FLU and the infusion of iNK cell therapy is usually between 40 and 84 hours. In some cases, the iNK cell therapy may be administered after the 84-hour timepoint. Dose adjustments for weight/creatinine are per institutional guidelines. Because of their deleterious effect on iNK cell-based therapy, corticosteroids as pre-medication for CY and FLU should be avoided unless considered necessary by the investigator and should not be administered within 24 hours before or after iNK cell therapy administration.

One iNK cell therapy product as described herein comprises CD38 knockout. The dosing scheme of an anti-CD38 mAb as exemplified in a combinational therapy with this iNK cell product (for example the QW doses on days prior to iNK cell infusion in Table 2) provides lymphodepletion through which the activated lymphocytes are eliminated for their upregulated CD38. A sufficient lymphodepletion through anti-CD38 mAb could provide an alternative conditioning process for the present iNK cell therapy, without or with minimal need of a CY/FLU-based lympho-conditioning procedure. Thus, in various embodiments wherein the course of treatment comprises administering to the subject an anti-CD38 monoclonal antibody, the method: (i) does not require CY/FLU-based lympho-conditioning; or (ii) requires a minimal need of CY/FLU-based lympho-conditioning. As used herein, the term “minimal need” in this context refers to: (i) a substantially lower dose that is about 90%, 80%, 70%, 60%, 50%, 40%, 30%, 20% or 10% of the dose that would be used without anti-CD38 lymphodepletion; and/or (ii) one or two fewer administrations, at the same or a lower dose, as compared to the three consecutive administrations of the CY/FLU-based lympho-conditioning.

Daratumumab: Daratumumab (Darzalex) is an anti-cancer drug that binds to CD38, a surface protein overexpressed in multiple myeloma cells. iNK cells engineered with enhanced CD16 efficacy and CD38 removed are resistant to CD38-targeted antibody-induced fratricide and more potently mediate anti-myeloma activity in combination with daratumumab. Daratumumab is administered by IV infusion at a dose of about 16 mg/kg actual body weight on a predetermined schedule, for example, starting on Day −11, then weekly (QW) for a total of 8 doses, then every 2 weeks (Q2W) for a total of 8 doses, then every 4 weeks (Q4W) until disease progression or unacceptable toxicity.

Because infusion-related reactions have been observed with daratumumab, and to minimize the potential impact of corticosteroids on NK cell function, pre- and post-infusion medications may be given following a general guideline as provided in Table 1. Within 1 week after starting daratumumab, antiviral prophylaxis is initiated to prevent herpes zoster reactivation and is continued for 3 months following treatment. Additional modifications to prophylaxis for infusion related reactions may be made based on case evaluation. In all cases, long-acting corticosteroids including dexamethasone are not administered.

TABLE 1
Daratumumab Pre-infusion and Post-infusion Medications

| Timepoint | Medication(s) |
|---|---|
| Pre-infusion | Oral acetaminophen 650-1000 mg prior |
| (1-3 hours prior | to each daratumumab dose |
| to daratumumab | Oral or IV diphenhydramine 25-50 mg, |
| infusion) | or equivalent, prior to each daratumumab |
| | dose |
| | Intermediate-acting corticosteroid (no |
| | long-acting corticosteroids should be |
| | administered) |
| | Prior to Day −11: methylprednisolone |
| | 100 mg, or equivalent, administered IV |
| | Prior to Day −4: methylprednisolone |
| | 60 mg, or equivalent, administered orally |
| | or IV |
| | For daratumumab doses administered |
| | between Day 1 and Day 29 of the iNK |
| | cell treatment cycle: methylprednisolone |
| | should not be administered. If an |
| | infusion-related reaction is observed |
| | following administration of daratumumab |
| | on Day −11 or Day −4, then |
| | methylprednisolone 60 mg orally should |
| | be administered prior to subsequent |
| | daratumumab infusions. |
| | For daratumumab doses administered |
| | outside of the iNK cell treatment cycle |
| | period: methylprednisolone 60 mg, or |
| | equivalent, administered orally or IV |
| | should be administered. |
| Post-infusion | For daratumumab doses administered |
| | between Day 1 and Day 29 of the iNK |
| | cell treatment cycle: post-infusion |
| | methylprednisolon should not be |
| | administered. If a delayed-onset |
| | infusion-related reaction is observed, |
| | then methylprednisolone 20 mg orally |
| | should be administered following |
| | subsequent infusions. |
| | For daratumumab doses administered |
| | outside of the iNK cell treatment cycle |
| | period: administer methylprednisolone |
| | 20 mg, or equivalent, on each of the 2 |
| | days following daratumumab infusion |
| | beginning the day after the infusion. |

For subjects with a history of chronic obstructive
pulmonary disease:
Consider prescribing post-infusion medication such as
short- and long-acting bronchodilators, and inhaled
corticosteroids.
Following the first four infusions, if the subject experiences
no major infusion-related reactions, these additional
inhaled post-infusion medications may be discontinued.

Elotuzumab (Combination Therapy 2): Elotuzumab is administered by IV infusion at a dose of about 10 mg/kg starting on Day −11, then QW for a total of 8 doses, and then Q2W until disease progression or unacceptable toxicity.

Because infusion-related reactions have been observed with elotuzumab, and to minimize the potential impact of corticosteroids on iNIK cell function, pre- and post-infusion medications may be given following a general guideline as provided in Table 2. Additional modifications to prophylaxis for infusion related reaction may be made based on case evaluation. It is recommended to initiate antiviral prophylaxis to prevent herpes zoster reactivation within 1 week after starting elotuzumab and to continue for 3 months following treatment. In all cases, long-acting corticosteroids such as dexamethasone are not administered.

TABLE 2
Elotuzumab Pre-infusion and Post-infusion Medications

| Timepoint | Medication(s) |
|---|---|
| Pre-infusion | Oral acetaminophen 650-1000 mg 45-90 |
| | minutes prior to each elotuzumab dose |
| | Oral or IV diphenhydramine 25-50 mg or |
| | equivalent 45-90 minutes prior to each |
| | elotuzumab dose |
| | Oral or IV ranitidine, 50 mg IV or |
| | 150 mg orally, or equivalent, 45-90 |
| | minutes prior to each elotuzumab dose |
| | Intermediate-acting corticosteroid |
| | (administered 1-3 hours prior to |
| | elotuzumab infusion; no long-acting |
| | corticosteroid should be administered) |
| | Prior to Day −11: methylprednisolone |
| | 100 mg, or equivalent, administered IV |
| | Prior to Day −4: 60 mg, or equivalent, |
| | administered orally or IV |
| | For elotuzumab doses administered between |
| | Day 1 and Day 29 of the iNK cell treatment |
| | cycle: methylprednisolone should not be |
| | administered. If an infusion-related reaction |
| | is observed following administration of |
| | elotuzumab on Day −11 or Day −4, then |
| | methylprednisolone 60 mg orally should |
| | be administered prior to subsequent |
| | elotuzumab infusions |
| | For elotuzumab doses administered outside |
| | of the iNK cell treatment cycle period: |
| | methylprednisolone 60 mg, or equivalent, |
| | administered orally or IV should be |
| | administered |
| Post-infusion | For elotuzumab doses administered between |
| | Day 1 and Day 29 of the iNK cell treatment |
| | cycle: post-infusion methylprenisolone should |
| | not be administered. If a delayed-onset |
| | infusion related reaction is observed, then |
| | methylprednisolone 20 mg orally should |
| | be administered following subsequent infusion. |
| | For elotuzumab doses administered outside of |
| | the iNK cell treatment cycle period: administer |
| | methylprednisolone 20 mg, or equivalent, on |
| | each of the 2 days following elotuzumab |
| | infusion beginning the day after the infusion. |

Permitted Therapy: Throughout the study, the investigator may prescribe any concomitant medications or treatment deemed necessary to provide adequate supportive care. Supportive care may include antibiotics, checkpoint inhibitors, analgesics, transfusions, growth factors, etc. Only irradiated blood products should be used to minimize the risk of transfusion associated GvHD. Biologically, radiation therapy may create a more immunogenic microenvironment that would enhance iNK cell anti-tumor activity. Subjects may receive palliative radiation therapy at any time and with schedules at the discretion of the investigator provided that the schedule of palliative radiation therapy does not interfere with protocol-specified schedule of activities.

Cautionary Therapy: Systemic corticosteroids are avoided during the treatment cycle, unless absolutely required, to avoid NK cell function inhibition.

Prohibited Therapy: Any antineoplastic agent for therapeutic intent other than protocol-directed study treatment(s) is prohibited with the following exceptions: (i) any prior therapy leading up to the administration of study treatment as described in the inclusion/exclusion criteria; (ii) anti-cancer therapy administered for disease progression following the iNK cell therapy treatment period; or (c) palliative radiotherapy.

Exploratory Analyses: Exploratory analyses of potential predictive and prognostic biomarkers associated with the mechanism of action of the iNK cell therapy and underlying disease immunobiology are performed. Biomarkers that correlate with clinical outcomes may be used for association with various indications and patient sub-populations. Changes in immune-related biomarkers, for example, pharmacodynamic biomarkers including, but not limited to, cytokines, iNK cell PK, and T-cell numbers and function, and any potential associations with dose-dependent safety and anti-tumor activity, in the peripheral blood and within tumors provide evidence for the biologic activity and efficacy of the iNK cell therapy.

Sample Collection and Analyses: Along with peripheral blood sampling, tumor samples, including but not limited to, bone marrow biopsies and aspirates are obtained from subjects prior to treatment, following initial treatment with the iNK cell therapy, and at later timepoints including, but not limited to, the time of disease progression or relapse. Peripheral blood samples are collected before and after cell infusion on days when the cells are administered to characterize the pharmacokinetics (PK) of the iNK cell therapy. iNK cell quantification may also be performed on tumor samples. These samples are useful for evaluating the ability of the iNK cell therapy to infiltrate sites of tumor, minimal residual disease (MRD) status, changes to the tumor microenvironment, and potential mechanisms of iNK cell resistance.

The collected peripheral blood samples are used for measurement of cytokine release syndrome (CRS) cytokines prior to iNK cell therapy administration and in the event of clinically suspected CRS. In addition to CRS, C reactive protein (CRP) and ferritin are also tested. The collected peripheral blood samples are also used for detecting alloimmunization to the iNK cell product human leukocyte antigen (HLA) by panel-reactive antibody and by assessments of T cell function, thereby evaluating immunogenicity of the iNK cell therapy. In addition, HLA and killer-cell immunoglobulin-like receptor (KIR) typing, and exploratory biomarker analysis which includes, but is not limited to: measurement of peripheral blood cytokine levels; measurement of protein biomarkers of disease progression and/or response to therapy; mAb PK, where applicable; and circulating tumor DNA/profiling, are conducted using the collected peripheral blood and/or serum samples.

Exploratory biomarker analysis is further performed on bone marrow biopsy/aspirate samples obtained while the subject is on study. The analysis includes, but is not limited to: MRD status by flow cytometry and/or DNA sequencing and clone identification; tumor somatic mutation profiling, e.g., tumor mutation burden, microsatellite instability, and/or ploidy analysis; tumor microenvironment characterization, e.g., tumor infiltrating T-cell characterization, tumor infiltrating innate immune cell characterization, and/or immune inhibitory molecule expression by tumor cells; and pre-treatment gene polymorphism and expression panels to explore correlation of alterations with outcome.

In addition, the collected peripheral blood and/or serum samples are used for monitoring one or more of the following: peripheral blood mononuclear cell (PBMC) functional characterization and immunophenotyping, e.g., T-cell subset analysis and determination of T-reg frequency; non-diagnostic exploratory MRD assessments; and peripheral blood immune cell functional assays.

Various assays for exploratory analyses include, but are not limited to, analysis of lymphocytes, T cell activation, T-cell receptor repertoire, cytokines associated with inflammation, circulating tumor DNA or MRD, cell of origin, and genes or gene signatures associated with tumor immunobiology. Exploratory analyses may involve extraction of DNA, cell-free DNA, or RNA; analysis of mutations, single nucleotide polymorphisms, and other genomic variants; and genomic profiling through use of next-generation sequencing of a comprehensive panel of genes.

Post-treatment follow-up visits will continue until up to 15 years following the initiation of study treatment administration or withdrawal of consent, whichever occurs first. Follow-up activities after iNK cell therapy treatment and prior to disease relapse or progression include clinical and laboratory assessments and disease response assessments.

### Example 2—Trial Patient Selection

Subjects are enrolled in two stages: a dose-escalation stage and a dose expansion stage. After the safety and tolerability are assessed to define the maximum tolerated dose (MTD) (or through the maximum assessed dose in the absence of a dose-limiting toxicity (DLT) defining the MTD) in the dose escalation stage, the dose-expansion stage is further evaluated for the safety and activity of the adoptive cell therapy. Patient recruitment is carried out by taking into account the following key inclusion/exclusion criteria for the clinical trial purposes of generating data that clearly demonstrate clinical benefit of the therapy without potentially confounded by residual treatment effects of prior therapy. As understood in the field, the trial subject inclusion/exclusion criteria for trial subject enrollment are not intended for setting limitations in a later approved label for said therapy.

### Inclusion Criteria—Combination Therapy 1—Multiple Myeloma (MM)

The patient must have a diagnosis of MM that has relapsed or progressed after at least two lines of therapies, including a proteasome inhibitor (e.g., bortezomib, carfilzomib, or ixazomib), an immunomodulatory drug (e.g., thalidomide, lenalidomide, or pomalidomide), anti-CD38 mAb therapy (e.g., daratumumab or isatuximab), and CAR-T therapies. Planned sequential therapy (e.g., induction therapy followed by stem-cell transplantation (SCT)) is considered one line of therapy. The patient must have measurable disease, which is defined by at least one of the following: (i) serum M-protein ≥1.0 g/dL; (ii) urine M-protein ≥200 mg/24 hours; and (iii) serum free light chain (FLC) level ≥10 mg/dL if serum M-protein <1.0 g/dL, urine M protein <200 mg/24 hours.

### Exclusion Criteria Specific to Combination Therapies Against Multiple Myeloma (MM)

Patients in either of the combination therapy regimens who meet any of the following criteria are excluded: (a) plasma cell leukemia defined as a plasma cell count >2000/mm³; (b) leptomeningeal involvement of MM; (c) receipt of any biological therapy, chemotherapy, or radiation therapy, except for palliative purposes, within two weeks prior to Day 1 or five half-lives, whichever is shorter; or any investigational therapy within 28 days prior to the first dose of monoclonal antibody (mAb); or (d) allergy or hypersensitivity to antibodies or antibody-related proteins.

### Example 3—A Phase I Study of FT576 as Monotherapy and in Combination with Daratumumab in Subjects with Relapsed/Refractory Multiple Myeloma

FT576 is a multiplexed-engineered NK cell therapy generated from a clonal master engineered induced pluripotent stem cell (iPSC) line, which can be used as a renewable source for the mass production of NK cells of uniform composition for off-the-shelf availability and broad patient access. FT576 is engineered with 4 modalities to combine multifaceted innate immunity with multiantigen-targeting capability: (1) high-affinity 158V, non-cleavable CD16 (hnCD16) Fc receptor for augmented antibody-dependent cellular cytotoxicity (ADCC); (2) IL-15/IL-15 receptor fusion that promotes NK cell persistence; (3) CD38 knockout to mitigate NK cell fratricide by CD38-directed monoclonal antibodies (mAbs) and to promote higher rates of glycolysis with improved metabolic fitness and resistance to oxidative stress found in the tumor microenvironment; and (4) a BCMA-directed CAR to target clonal plasma cells. These modalities are designed to enhance the potency and persistence of FT576 as well as enable multiantigen targeting. The BCMA-directed CAR used herein comprises an Ig kappa chain variable leader peptide, a BCMA scFV, a human IgG4 Fe, a transmembrane domain derived from NKG2D, a co-stimulatory domain derived from 2B4, and a signaling domain comprising the native or modified ITAM1 (immunoreceptor tyrosine-based activation motif) of CD3ζ, wherein the amino acid sequence of such a structure is of at least about 85%, about 90%, about 95%, about 96%, about 97%, about 98%, or about 99% identity to SEQ ID NO: 7. In particular embodiments, the BCMA scFV is characterized in that the antigen-binding domain comprises a variable heavy chain (VH), said VH comprising: a heavy chain complementary determining region 1 (H-CDR1) with at least 80% sequence identity (e.g., 90% or 100%) to SEQ ID NO: 8 (GFTFSRYW), a heavy chain complementary determining region 2 (H-CDR2) with at least 80% sequence identity (e.g., 90% or 100%) to SEQ ID NO: 9 (INPSSSTI), and a heavy chain complementary determining region 3 (H-CDR3) with at least 80% sequence identity (e.g., 90% or 100%) to SEQ ID NO: 10 (ASLYYDYGDAYDY); and a variable light chain (VL), said VL comprising: a light chain complementary determining region 1 (L-CDR1) with at least 80% sequence identity (e.g., 90% or 100%) to SEQ ID NO: 11 (QSVESN), a light chain complementary determining region 2 (L-CDR2) with at least 80% sequence identity (e.g., 90% or 100%) to SEQ ID NO: 12 (SAS), and a light chain complementary determining region 3 (L-CDR3) with at least 80% sequence identity (e.g., 90% or 100%) to SEQ ID NO: 13 (QQYNNYPLT). In some embodiments, the BCMA directed CAR comprises the amino acid sequence of SEQ ID NO: 7.

SEQ ID NO: 7
MDFQVQIFSFLLISASVIMSREVQLVESGGGLVQPGGSLRLSCAASGFT
FSRYWESWVRQAPGKGLVWVGEINPSSSTINYAPSLKDKFTISRDNAKN
TLYLQMNSLRAEDTAVYYCASLYYDYGDAYDYWGQGTLVTVSSGSTSGS
GKPGSGEGSTKGEIVMTQSPATLSVSPGERATLSCKASQSVESNVAWYQ
QKPGQAPRALIYSASLRFSGIPARFSGSGSGTEFTLTISSLQSEDFAVY
YCQQYNNYPLTFGAGTKLELKESKYGPPCPSCPAPEFEGGPSVFLFPPK
PKDTLMISRTPEVTCVVVDVSQEDPEVQFNWYVDGVEVHNAKTKPREEQ
FQSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSPGKSNLFVASWIAVMIIFRIGMAVAIFCCFFFPSWRRKRKEKQSETS
PKEFLTIYEDVKDLKTRRNHEQEQTFPGGGSTIYSMIQSQSSAPTSQEP
AYTLYSLIQPSRKSGSRKRNHSPSENSTIYEVIGKSQPKAQNPARLSRK
ELENFDVYSRVKFSRSADAPAYKQGQNQLYNELNLGRREEYDVLDKRRG
RDPEMGGKPRRKNPQEGLYNELQKDKMAEAYSEIGMKGERRRGKGHDGL
YQGLSTATKDTYDALHMQALPPR
(BCMA-CAR; Leader peptide + BCMA scFV +
IgG4 Fc + NKG2D TM + 2B4 + CD32)

Multiple myeloma (MM) is a cancer that forms in a type of white blood cell referred to as a plasma cell. Cancerous plasma cells accumulate in the bone marrow and crowd out healthy blood cells leading to complications. This study evaluates the clinical activity of iNK cells in combination with a monoclonal antibody (mAb) in subjects with advanced hematologic malignancies, specifically r/r MM, which remains an area of significant unmet medical need, and which is potentially amenable to NK cell-based therapies. ADCC resulting from engagement of the Fc portion of the mAb with CD16 on NK cells is a major mechanism of action contributing to the clinical efficacy of currently approved mAbs targeting CD38 (e.g., daratumumab, Darzalex USPI) and SLAMF7 (e.g., elotuzumab, Empliciti USPI) in MM. Given that NK cells utilize ADCC as a mechanism of anti-tumor activity, and with the expression of hnCD16 to enhance ADCC, said iNK cell therapy is used in combination with mAbs as a way of enhancing ADCC.

Study Design and Methods: This is a multicenter, Phase I clinical trial of FT576 in patients with R/R MM. The primary objectives are to determine the recommended Phase II dose of FT576, given as single or fractionated doses, as monotherapy and in combination with daratumumab in R/R MM and to evaluate safety and tolerability. Key secondary objectives include antitumor activity and pharmacokinetics as monotherapy and combined with daratumumab in R/R MM. Exploratory objectives include characterization of FT576 pharmacodynamics, assessment of minimal residual disease, and characterization of the tumor microenvironment in pre- and post-treatment.

The dose-escalation stage of the trial has the following 4 arms (Table 3): single-dose FT576 monotherapy on Day 1 (Regimen A); multidose FT576 monotherapy on Days 1 and 15 (Regimen A1); single-dose FT576+daratumumab on Day 1 (Regimen B); and multidose FT576+daratumumab on Days 1 and 15 (Regimen B1). Up to 5 dose levels of FT576 ranging from 100 million to 1.5 billion cells per dose will be tested using a modified toxicity probability interval (mTPI) dose-escalation design. Daratumumab will be administered per approved dose and schedule. Conditioning chemotherapy consisting of 3 consecutive days of fludarabine and cyclophosphamide will be administered prior to the first dose of FT576. Key inclusion criteria include R/R disease after standard approved therapies and measurable disease. Prior BCMA, including CAR T cell, and anti-CD38-targeted therapies are permitted.

TABLE 3
Dose-escalation stage of the FT576 trial study

| | FT576 |
|---|---|
| | (hnCD16 + IL15RF + |
| | CD38KO + CAR-BCMA) |
| Treatment | FT576 +/− daratumumab |
| Setting | Relapsed/Refractory MM |
| Dose/Schedule | 1- and 2-dose treatment schedules × 1 cycle; |
| | Second cycle subject to FDA consent |
| | DL1 = 100M |
| | DL2 = 300M |
| | DL3 = 1B |
| | DL4 = 1.5B |

The dose-escalation stage determines appropriate dose levels of the adoptive cell therapy not exceeding the maximum tolerated dose (MTD) as a monotherapy or in combination with monoclonal antibodies (mAbs) for further assessment, and determines a recommended dose for use in combination with each of the tested mAbs in dose expansion.

Dose escalation proceeds independently among iNK cell monotherapies and combinational therapies in order to identify the MTD/MAD of each of the therapeutic regimens. Initiation of Combination 1 (iNK cells+daratumumab) is contingent on clearance of the first iNK cell monotherapy dose escalation cohort at a dose level corresponding to that of the cleared iNK cell monotherapy dose escalation cohort. Dose escalation will proceed independently between iNK cell monotherapy and combination through DL2 dose escalation, at which time dose escalation is paused to review safety, preliminary efficacy, and iNK cell PK data.

A dose-limiting toxicity (DLT) is defined as any adverse event (AE) that is at least possibly related to iNK cell therapy that occurs after the first iNK cell infusion through the end of the DLT assessment period on Day 29, and with extension of the DLT assessment period beyond Day 29 to allow for AE recovery as defined below, that meets one of the following criteria based on the National Cancer Institute Common Terminology Criteria for Adverse Events, Version 5.0 (NCI CTCAE, v5.0) or the American Society for Transplantation and Cellular Therapy (ASTCT) Consensus Grading Guidelines for Cytokine Release Syndrome and Neurological Toxicity Associated with Immune Effector Cells (Lee et al. 2019). Grading of acute GvHD is based on the Center for International Blood and Marrow Transplant Research (CIBMTR) acute GvHD scoring scale. Grading of laboratory AEs is assessed relative to baseline laboratory values defined as the last assessment prior to the start of protocol-defined study medication. Table 4 is used for assessing severity of AEs that are not listed in the NCI CTCAE, v5.0.

TABLE 4
Severity Grading Scale for Adverse Events Not Listed in NCI CTCAE

| | |
|---|---|
| Grade 1 | Mild: asymptomatic or mild symptoms; |
| | clinical or diagnostic observations only; |
| | intervention not indicated |
| Grade 2 | Moderate: minimal, local or noninvasive intervention indicated; |
| | limiting age-appropriate instrumental activities of daily living |
| Grade 3 | Severe: severe or medically significant but not immediately |
| | life-threatening; hospitalization or |
| | prolongation of hospitalization |
| | indicated; disabling; limiting self-care |
| | activities of daily living |
| Grade 4 | Life-threatening: life-threatening consequences; |
| | urgent intervention indicated |
| Grade 5 | Death: Death-related AE |

For all patients, a DLT includes, but is not limited to, any non-hematologic AE Grade ≥4 (e.g., a Grade 4 infusion-related reaction); Grade 3 pulmonary or cardiac AE of any duration; any non-hematologic AE Grade 3 (with the exception of Grade 3 pulmonary or cardiac toxicity of >72 hours' duration); Grade 3 immune cell associated neurotoxicity syndrome (ICANS) of any duration; and any Grade ≥2 acute GvHD requiring systemic steroid administration and without resolution to Grade 1 within 7 days. However, fever associated with cytokine release syndrome (CRS) that occurs in the context of Grade <3 CRS will not be considered a DLT. Additional exceptions that are not considered DLTs, include: Grade 3 renal or hepatic AE lasting <7 days; Grade 3 laboratory abnormality, unless otherwise specified, that is asymptomatic and determined by the treating investigator not to be clinically significant; and Grade 3 fatigue lasting ≤3 days.

For patients receiving the iNK cell monotherapy, a hematologic AE includes prolonged myelosuppression through 42 days following administration of the first dose of the iNK cell therapy, defined as Grade 4 neutropenia refractory to granulocyte-colony stimulating factor (G-CSF) in the absence of the following: morphologic evidence of acute leukemia, as assessed by circulating blasts in the peripheral blood or leukemic blasts in a bone marrow biopsy and/or evidence of recovering hematopoiesis, as defined by bone marrow >5% cellularity.

For patients receiving combination therapy, a hematologic AE includes any Grade ≥3 hematologic AE. The following are exceptions and therefore will not be considered DLTs: (i) Grade 3-4 neutropenia refractory to G-CSF in the absence documented infections that improves to Grade ≤2 or to ≤80% of baseline, whichever is lower, within 21 days of the neutrophil count nadir; (ii) Grade 3-4 anemia in the absence of ongoing red blood transfusion and association with clinical signs and symptoms that improves to Grade ≤2 or to ≥80% of baseline, whichever is lower, within 21 days of the hemoglobin concentration nadir; and (iii) Grade 3-4 thrombocytopenia in the absence of Grade ≥2 bleeding that improves to Grade ≤2 or to ≥80% of baseline, whichever is lower, within 21 days of the platelet count nadir.

### Example 4—Dose-Expansion Stage

The objectives of dose expansion are to further assess safety and tolerability of the adoptive cell therapy as a monotherapy in subjects with r/r MM (relapsed/refractory Multiple Myeloma), and to identify clinical activity signals to guide and support future development. Enrollment in dose expansion occurs independently among the regimens (1- or 2-dose iNK cell monotherapy, and 1- or 2-dose combination therapy).

The doses of the adoptive cell therapy for dose expansion with the three regimens are each determined based on the clinical and available pharmacokinetic (PK) and pharmacodynamic (PD) data from dose escalation and do not exceed the MTD or the MAD for that regimen. Enrollment into a dose-expansion cohort may begin once a given dose level is cleared in dose escalation. For each regimen, more than one dose-expansion cohort of subjects may be opened to: (i) more fully characterize safety/tolerability at a given dose level, including evaluation of alternate doses and schedules of the adoptive cell therapy to optimize safety and tolerability, provided the dose does not exceed the MTD or MAD; and (ii) more fully characterize clinical activity in a specific indication or patient population defined by a specific regimen.

For subjects with r/r MM, disease response is assessed through analysis of bone marrow biopsy/aspirate and peripheral blood for enumeration of leukemic blasts, and classified into the best of the following response categories: stringent complete response (sCR), complete response (CR), very good partial response (VGPR), partial response (PR), minimal response (MR), stable disease (SD), or progressive disease (PD) using the criteria shown in Table 5.

TABLE 5
Criteria in Multiple Meyloma, Including Criteria for Minimal Residual
Disease Response Criteria
MRD Criteria (requires a CR, as defined below)

| | |
|---|---|
| Sustained | MRD negativity in the marrow (NGF or NGS, or both) |
| MRD- | and by imaging as defined below, confirmed minimum |
| negative | of 1 year apart. Subsequent evaluations can be used to |
| | further specify the duration of negativity (e.g., MRD- |
| | negative at 5 years) |
| Flow | Absence of phenotypically aberrant clonal plasma cells by |
| MRD- | NGF on bone marrow aspirates using the EuroFlow |
| negative | standard operation procedure for MRD detection in |
| | multiple myeloma (or validated equivalent method) with |
| | a minimum sensitivity of 1 in 10⁵ nucleated cells or higher |
| Sequencing | Absence of clonal plasma cells by NGS on bone marrow |
| MRD- | aspirate in which presence of a clone is defined as less |
| negative | than two identical sequencing reads obtained after DNA |
| | sequencing of bone marrow aspirates using the |
| | LymphoSIGHT platform (or validated equivalent |
| | method) with a minimum sensitivity of 1 in 10⁵ |
| | nucleated cells or higher |
| Imaging | MRD negativity as defined by NGF or NGS plus |
| plus MRD- | disappearance of every area of increased tracer uptake |
| negative | found at baseline or a preceding PET/CT or decrease |
| | to less mediastinal blood pool SUV or decrease to less |
| | than that of surrounding normal tissue |

Response Criteria

| | |
|---|---|
| Stringent | CR as defined below plus normal FLC ratio and absence |
| complete | of clonal cells in bone marrow biopsy by immuno- |
| response | histochemistry (κ/λ ratio ≤4:1 or ≥1:2 for κ and λ |
| (sCR) | patients, respectively, after counting ≥100 plasma cells) |
| Complete | Negative immunofixation on the serum and urine and |
| response | disappearance of any soft tissue plasmacytomas and |
| (CR) | <5% plasma cells in bone marrow aspirates |
| Very good | Serum and urine M-protein detectable by immuno- |
| partial | fixation but not on electrophoresis or ≥90% reduction |
| response | in serum M-protein plus urine M-protein level <100 |
| (VGPR) | mg per 24 hours |
| Partial | ≥50% reduction of serum M-protein plus reduction in |
| response | 24-h urinary M-protein by ≥90% or to <200 mg per 24 h; |
| (PR) | If the serum and urine M-protein are unmeasurable, |
| | a ≥50% decrease in the difference between involved |
| | and uninvolved FLC levels is required in place of the |
| | M-protein criteria; |
| | If serum and urine M-protein are unmeasurable, and |
| | serum-free light assay is also unmeasurable, ≥50% |
| | reduction in plasma cells is required in place of M- |
| | protein, provided baseline bone marrow plasma-cell |
| | percentage was ≥30%. In addition to these criteria, if |
| | present at baseline, a ≥50% reduction in the size (SPD) ⁱ |
| | of soft tissue plasmacytomas is also required. |
| Minimal | ≥25% but ≤49% reduction of serum M-protein and |
| response | reduction in 24-h urine M-protein by 50-89%. In |
| | addition to the above listed criteria, if present at baseline, |
| | a ≥50% reduction in the size (SPD) of soft tissue |
| | plasmacytomas is also required. |
| Stable | Not recommended for use as an indicator of response; |
| disease | stability of disease is best described by providing the |
| (SD) | time-to-progression estimates. Not meeting criteria |
| | for CR, VGPR, PR, MR, or PD. |
| Progressive | Any one or more of the following criteria: |
| disease | Increase of 25% from lowest confirmed response value |
| (PD) | in one or more of the following criteria: |
| | Serum M-protein (absolute increase must be >0.5 g/dL); |
| | Serum M-protein increase ≥1 g/dL, if the lowest |
| | M-component was ≥5 g/dL; |
| | Urine M-protein (absolute increase must be ≥200 mg/24 h); |
| | In patients without measurable serum and urine M-protein |
| | levels, the difference between involved and uninvolved |
| | FLC levels (absolute increase must be >10 mg/dL); |
| | In patients without measurable serum and urine M-protein |
| | levels and without measurable involved FLC levels, bone |
| | marrow plasma-cell percentage irrespective of baseline |
| | status (absolute increase must be ≥10%); |
| | Appearance of a new lesion(s), ≥50% increase from nadir |
| | in SPD of >1 lesion, or ≥50% increase in the longest |
| | diameter of a previous lesion >1 cm in short axis; |
| | ≥50% increase in circulating plasma cells (minimum of |
| | 200 cells per μL) if this is the only measure of disease |
| Clinical | Clinical relapse requires one or more of the following |
| relapse | criteria: |
| | Direct indicators of increasing disease and/or end organ |
| | dysfunction (CRAB features) related to the underlying |
| | clonal plasma-cell proliferative disorder. It is not used in |
| | calculation of time to progression or progression-free |
| | survival but is listed as something than can be reported |
| | optionally or for use in clinical practice; |
| | Development of new soft tissue plasmacytomas or bone |
| | lesions (osteoporotic fractures do not constitute progression); |
| | Definite increase in the size of existing plasmacytomas |
| | or bone lesions. A definite increase is defined as a 50% |
| | (and ≥1 cm) increase as measured serially by the SPD of |
| | the measurable lesion; |
| | Hypercalcemia (>11 mg/dL); |
| | Decrease in hemoglobin of ≥2 g/dL not related to therapy |
| | or other non-meyloma-related conditions; |
| | Rise in serum creatinine by 2 mg/dL or more from the |
| | start of the therapy and attribute to myeloma; |
| | Hyperviscosity related to serum paraprotein |
| Relapse from | Any one or more of the following criteria: |
| complete | Reappearance of serum or urine M-protein by immuno- |
| response | fixation or electrophoresis; |
| (to be used | Development of ≥5% plasma cells in the bone marrow; |
| only if the | Appearance of any other sign of progression (i.e., new |
| end point is | plasmacytoma, lytic bone lesion, or hypercalcemia see |
| disease-free | above) |
| survival) | |
| Relapse | Any one or more of the following criteria: |
| from MRD | Loss of MRD negative state (evidence of clonal plasma |
| neative | cells on NGF or NGS, or positive imaging study for |
| (to be used | recurrence of myeloma); |
| only if the | Reappearance of serum or urine M-protein by immuno- |
| end point | fixation or electrophoresis; |
| is disease-free | Development of ≥5% clonal plasma cells in the bone marrow; |
| survival) | Appearance of any other sign of progression (i.e., new |
| | plasmacytoma, lytic bone lesion, or hypercalcemia) |

ASCT, autologous stem-cell transplantation; CR, complete response; CRAB features, calcium elevation, renal failure, anemia, lytic bone lesions; CT, computed tomography (scan); ¹⁸F-FDG-PET, ¹⁸F-fluorodeoxyglucose-PET (scan); FCM, flow cytometry; FLC, free light chain; IMWG, International Myeloma Working Group; M-protein, meyloma protein; MFC, multiparameter flow cytometry; MR, minimal response; MRD; minimal residual disease; MRI, magnetic resonance imaging (scan); NGF, next-generation flow; NGS, next-generation sequencing; PD, progressive disease; PET/CT, positron emission tomography/computed tomography (scan); PR, partial response; sCR, stringent complete response; SD, stable disease; SPD, sum of the products of the maximal prependicular diameters of measured lesions; SUVₘₐₓ, maximum standardized uptake value; VGPR, very good partial response.

As part of the best overall response, the secondary endpoint of ORR is used to summarize the proportion of subjects achieving a response of PR or better by regimen and dose level. In addition, the exact 95% CI is determined. For subjects with an sCR or CR response, the proportion of subjects: with a MRD negative response, with a sustained MRD-negative response for at least 1 year, and MRD negative response with imaging based on the IMWG MRD criteria are summarized descriptively along with the 95% CI by regimen and dose level as exploratory endpoints. The time-to-event endpoints include duration of response (DOR), PFS, RFS from CR, OS as secondary endpoints and time to MRD-negative response, and RFS from MRD-negative response as exploratory endpoints.

With evidence of clinical benefit demonstrated, an additional treatment cycle consisting of lympho-conditioning followed by the adoptive cell therapy, following the same schedule as the first treatment cycle may be considered based on a review of clinical data demonstrating evidence of clinical benefit.

Thus, in some embodiments, the course of treatment comprises: (i) one cycle of administration of the adoptive cell therapy product; (ii) one, two or three cycles of administration of the adoptive cell therapy product, with each cycle comprises one or two doses at a dose frequency of 1 dose per 15 days; or (iii) more than three cycles of administration of the adoptive cell therapy product over an extended period of time at a dose frequency based on clinical assessment of disease response, as set forth above. In some embodiments, within the context of a treatment regimen comprising multiple doses of cell therapy product, a cycle of treatment is said to be completed after each dose of cells is infused at a predetermined timepoint over a predetermined duration; and in some embodiments, more than one such cycle of treatment may be needed as medically determined.

As of data cutoff in July 2022, 9 patients with R/R MM were treated and evaluable for safety and efficacy, in the first 2 dose levels of Regimen A (multi-dose monotherapy; n=6) and in the first dose level of Regimen B (single dose+anti-CD38; n=3). No dose-limiting toxicities, and no events of any grade of cytokine release syndrome (CRS), immune effector cell-associated neurotoxicity syndrome (ICANS), or graft-versus-host disease (GvHD) were observed. There is evidence of anti-myeloma activity per IMWG 2016 based on the initial data (Tables 6-8).

TABLE 6
All regimen

| | | Regimen B: |
|---|---|---|
| | Regimen A: | FT576 + |
| | FT576 | Daratumumab |
| | (N = 5) | (N = 3) |
| Best Overall Response | | |
| Stringent complete | 0 | 0 |
| response (sCR) | | |
| CR | 0 | 0 |
| Very good PR | 1 (20.0) | 0 |
| PR | 0 | 0 |
| MR | 0 | 1 (33.3) |
| SD | 3 (60.0) | 2 (66.7) |
| PD | 1 (20.0) | 0 |
| Clinical Relapse | 0 | 0 |
| CR + sCR + | 1 (20.0) | 1 (33.3) |
| VGPR + PR + MR | | |

TABLE 7
Regimen A

| | 1 × 10⁸ | 3 × 10⁸ | All Dose |
|---|---|---|---|
| | cells | cells | Cohorts |
| | (N = 3) | (N = 2) | (N = 5) |
| Best Overall Response | | | |
| Stringent complete | 0 | 0 | 0 |
| response (sCR) | | | |
| CR | 0 | 0 | 0 |
| Very good PR | 0 | 1 (50.0) | 1 (20.0) |
| PR | 0 | 0 | 0 |
| MR | 0 | 0 | 0 |
| SD | 2 (66.7) | 1 (50.0) | 3 (60.0) |
| PD | 1 (33.3) | 0 | 1 (20.0) |
| Clinical Relapse | 0 | 0 | 0 |
| CR + sCR + | 0 | 1 (50.0) | 1 (20.0) |
| VGPR + PR + MR | | | |

TABLE 8
Regimen B

| | 1 × 10⁸ | All Dose |
|---|---|---|
| | cells | Cohorts |
| | (N = 3) | (N = 3) |
| Best Overall Response | | |
| Stringent complete | 0 | 0 |
| response (sCR) | | |
| CR | 0 | 0 |
| Very good PR | 0 | 0 |
| PR | 0 | 0 |
| MR | 1 (33.3) | 1 (33.3) |
| SD | 2 (66.7) | 2 (66.7) |
| PD | 0 | 0 |
| Clinical Relapse | 0 | 0 |
| CR + sCR + | 1 (33.3) | 1 (33.3) |
| VGPR + PR + MR | | |

sCR: stringent complete response: CR: complete response; VGPR: very good partial response; PR: partial response; MR: minimal response; SD: stable disease; or PD: progressive disease.

### Example 5—Retreatment Following Progression

With demonstrated evidence of clinical benefit, subjects whose disease has an objective response to the iNK cell therapy and later relapse or progress are eligible to receive a second course of treatment. An objective response achieved with a second course of treatment with the iNK cell therapy plus mAbs supports a longer treatment duration, e.g., to achieve deeper responses that may drive longer durations of clinical benefit and/or incorporation of the iNK cell therapy with mAb combination retreatment as part of the standard dosing schedule. Treatment-emergent changes in the tumor microenvironment are characterized for understanding of the potential mechanisms of resistance to treatment with the iNK cell therapy plus mAbs. Evidence of clinical benefit includes, but is not limited to: (i) absence of signs and symptoms, including worsening laboratory values, indicating unequivocal disease progression; (ii) no decline in Eastern Cooperative Oncology Group (ECOG) Performance Status; and/or (iii) no clear evidence of progressive disease. Exceptions may be made in cases where pseudoprogression due to the influx of immune cells into tumor sites are suspected, provided absence of signs and symptoms (including worsening of laboratory values) indicating unequivocal clinical disease progression, and an absence of tumor progression at critical anatomical sites where compromised organ function may increase the acute risk of severe and/or irreversible disability or death.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating a subject suitable for an adoptive cell therapy,
(i) wherein the subject has a hematological cancer;
(ii) wherein the method comprises administering to the subject at least a first cycle of an adoptive cell therapy product, with the first cycle comprising one or more doses of the adoptive cell therapy product administered in a first effective amount at a preselected frequency, and with an option of administering one or more additional cycles with one or more doses in a second effective amount, during a course of treatment over a period of time;
(iii) wherein the first and the second effective amounts are the same or different; and
(iv) wherein the product comprises an engineered natural killer (NK) lineage cell comprising exogenous CD16 expression, IL15RF expression, CD38 knockout and a BCMA directed CAR (chimeric antigen receptor).

2. The method of claim 1, wherein the hematological cancer comprises:
(i) multiple myeloma (MM); or
(ii) relapsed or refractory MM (r/r MM).

3. The method of claim 1, wherein the course of treatment further comprises administering to the subject an effective amount of a tumor-targeting, ADCC-capable monoclonal antibody (mAb).

4. The method of claim 1, wherein the course of treatment further comprises administering to the subject initial doses of a monoclonal antibody in an effective amount at a starting time prior to the first cycle of administering the adoptive cell therapy product, wherein the monoclonal antibody is an anti-CD38 monoclonal antibody.

5. The method of claim 4, wherein the starting time is about 8-12 days prior to the first cycle of administering the adoptive cell therapy product, and wherein the initial doses of the monoclonal antibody comprise 6-10 weekly (QW) doses, and optionally followed by 6-10 bi-weekly (Q2W±1 day) doses thereafter.

6. The method of claim 5, wherein the course of treatment further comprises administering to the subject the same anti-CD38 monoclonal antibody in an effective amount for 8 bi-weekly doses (Q2W±1 day) following the initial doses of administration of the monoclonal antibody.

7. The method of claim 6, further comprising administering to the subject one dose of the same anti-CD38 monoclonal antibody every four weeks (Q4W±1 day) in an effective amount until dose termination.

8. The method of any one of claims 4-7, wherein the anti-CD38 monoclonal antibody comprises daratumumab.

9. The method of any one of claims 1-8, further comprising administering to the subject at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time.

10. The method of claim 9, wherein the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m².

11. The method of claim 9, wherein the duration between is: (i) about 40-84 hours; or (ii) about 3 days.

12. The method of any one of claims 4-6, wherein the course of treatment comprises administering to the subject an anti-CD38 monoclonal antibody, and wherein the method: (i) does not require lympho-conditioning; or (ii) requires a minimal need of lympho-conditioning.

13. The method of claim 12, wherein the lympho-conditioning is CY/FLU-based.

14. The method of claim 1, wherein the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR.

15. The method of claim 1, wherein the effective amount of the adoptive cell therapy product in a dose is about 5×10⁷ cells to about 3×10⁹ cells.

16. The method of claim 15, wherein the effective amount of the adoptive cell therapy product in each dose is about 1×10⁸, 3×10⁸, 10×10⁸, or about 1.5×10⁹ cells.

17. The method of claim 8, wherein:
(i) daratumumab is in an amount of about 15 mg/kg to about 17 mg/kg; or
(ii) daratumumab is in an amount of about 16 mg/kg.

18. The method of claim 1, wherein the subject suitable for the adoptive cell therapy product has a diagnosis of MM comprising:
(i) a measurable disease; or
(ii) having no complete remission (CR), or having relapse, or evidence of progressive disease (PD) after one or more prior lines of therapy.

19. The method of claim 18, wherein the prior lines of therapy for MM, or relapsed or refractory MM comprise:
(a) a chemotherapy, an immunochemotherapy, hematopoietic stem cell transplantation, chimeric antigen receptor (CAR) T-cell therapy, or any combination thereof; or
(b) a proteasome inhibitor, an anti-CD38 antibody, an anti-SLAMF7 antibody, an immunomodulatory drug, a stem-cell transplantation (SCT), or any combination thereof, or
(c) bortezomib, carfilzomib, ixazomib, daratumumab, isatuximab, elotuzumab, thalidomide, lenalidomide, pomalidomide, or any combination thereof, or
(d) Carfilzomib.

20. The method of claim 1, wherein the method comprises administering (i) one cycle of the adoptive cell therapy product over about 29 days with 1 or 2 doses per cycle; (ii) two or more cycles of the adoptive cell therapy product, with each cycle comprising 1 or 2 doses; (iii) single dose per cycle over about 29 days in one cycle, with one or more cycles of the adoptive cell therapy product over an extended period of time based on clinical assessment of disease response; or (iv) 2 doses in one cycle over about 29 days, with one or more cycles of the adoptive cell therapy product over an extended period of time based on clinical assessment of disease response.

21. The method of any one of claims 1-20, wherein the administering of the adoptive cell therapy product is (i) via intravenous infusion, and/or (ii) at a site of an outpatient setting; and/or wherein each dose of the adoptive cell therapy product is cryopreserved, and then thawed prior to administering.

22. The method of any one of claims 1-21, wherein the BCMA directed CAR comprises:
(i) a variable heavy chain (VH) and a variable light chain (VL), wherein:
(a) said VH comprises: a heavy chain complementary determining region 1 (H-CDR1) with at least 80% sequence identity to SEQ ID NO: 8 (GFTFSRYW), a heavy chain complementary determining region 2 (H-CDR2) with at least 80% sequence identity to SEQ ID NO: 9 (INPSSSTI), and a heavy chain complementary determining region 3 (H-CDR3) with at least 80% sequence identity to SEQ ID NO: 10 (ASLYYDYGDAYDY); and
(b) a variable light chain (VL), said VL comprising: a light chain complementary determining region 1 (L-CDR1) with at least 80% sequence identity to SEQ ID NO: 11 (QSVESN), a light chain complementary determining region 2 (L-CDR2) with at least 80% sequence identity to SEQ ID NO: 12 (SAS), and a light chain complementary determining region 3 (L-CDR3) with at least 80% sequence identity to SEQ ID NO: 13 (QQYNNYPLT); or
(ii) an amino acid sequence that is of at least about 99%, 98%, 96%, 95%, 90%, 85%, or 80% identity to SEQ ID NO: 7.

23. A composition comprising an engineered natural killer (NK) lineage cell for use in treating a hematological cancer in a subject,
(i) wherein said engineered NK lineage cell comprises exogenous CD16 expression, IL15RF expression, CD38 knockout and a BCMA directed CAR (chimeric antigen receptor);
(ii) wherein said use comprises a course of treatment comprising at least a first cycle of an adoptive cell therapy product comprising said engineered NK lineage cell, with the first cycle comprising one or more doses of the adoptive cell therapy product in a first effective amount at a preselected frequency, and with an option of one or more additional cycles with one or more doses in a second effective amount, over a period of time; and
(iii) wherein the first and the second effective amounts are the same or different.

24. The composition for use of claim 23, wherein the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR.

25. The composition for use of claim 23, wherein the course of treatment further comprises an effective amount of a tumor-targeting, ADCC-capable monoclonal antibody (mAb).

26. The composition for use of claim 23, wherein the course of treatment further comprises initial doses of a monoclonal antibody provided in an effective amount at a starting time prior to the first cycle of the adoptive cell therapy product, wherein the monoclonal antibody is an anti-CD38 monoclonal antibody.

27. The composition for use of claim 26, wherein the anti-CD38 monoclonal antibody comprises daratumumab.

28. The composition for use of any one of claims 23-27, wherein the course of treatment further comprises at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time.

29. The composition for use of claim 28, wherein the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m².

30. The composition for use of any one of claims 23-29, wherein the BCMA directed CAR comprises:
(i) a variable heavy chain (VH) and a variable light chain (VL), wherein:
(a) said VH comprises: a heavy chain complementary determining region 1 (H-CDR1) with at least 80% sequence identity to SEQ ID NO: 8 (GFTFSRYW), a heavy chain complementary determining region 2 (H-CDR2) with at least 80% sequence identity to SEQ ID NO: 9 (INPSSSTI), and a heavy chain complementary determining region 3 (H-CDR3) with at least 80% sequence identity to SEQ ID NO: 10 (ASLYYDYGDAYDY); and
(b) a variable light chain (VL), said VL comprising: a light chain complementary determining region 1 (L-CDR1) with at least 80% sequence identity to SEQ ID NO: 11 (QSVESN), a light chain complementary determining region 2 (L-CDR2) with at least 80% sequence identity to SEQ ID NO: 12 (SAS), and a light chain complementary determining region 3 (L-CDR3) with at least 80% sequence identity to SEQ ID NO: 13 (QQYNNYPLT); or
(ii) an amino acid sequence that is of at least about 99%, 98%, 96%, 95%, 90%, 85%, or 80% identity to SEQ ID NO: 7.

31. Use of an engineered natural killer (NK) lineage cell in the manufacture of an adoptive cell therapy product for treating a hematological cancer,
(i) wherein said engineered NK lineage cell comprises exogenous CD16 expression, IL15RF expression, CD38 knockout and a BCMA directed CAR (chimeric antigen receptor);
(ii) wherein said adoptive cell therapy product is for use in a course of treatment comprising at least a first cycle of the adoptive cell therapy product, with the first cycle comprising one or more doses of the adoptive cell therapy product in a first effective amount at a preselected frequency, and with an option of one or more additional cycles with one or more doses in a second effective amount, over a period of time; and
(iii) wherein the first and the second effective amounts are the same or different.

32. The use of claim 31, wherein the engineered NK lineage cell is derived from an engineered induced pluripotent stem cell (iPSC) comprising a polynucleotide encoding an exogenous CD16, a polynucleotide encoding IL15RF, CD38 knockout, and a BCMA-directed CAR.

33. The use of claim 31, wherein the course of treatment further comprises an effective amount of a tumor-targeting, ADCC-capable monoclonal antibody (mAb).

34. The use of claim 31, wherein the course of treatment further comprises initial doses of a monoclonal antibody provided in an effective amount at a starting time prior to the first cycle of the adoptive cell therapy product, wherein the monoclonal antibody is an anti-CD38 monoclonal antibody.

35. The use of claim 34, wherein the anti-CD38 monoclonal antibody comprises daratumumab.

36. The use of any one of claims 31-35, wherein the course of treatment further comprises at least one daily dose of one or more chemotherapeutic agents prior to the first cycle of the adoptive cell therapy product, wherein a duration between the administration of a last daily dose of the one or more chemotherapeutic agents and the first cycle of the adoptive cell therapy product comprises a specified period of time.

37. The use of claim 36, wherein the one or more chemotherapeutic agents comprise cyclophosphamide (CY) and fludarabine (FLU); and optionally wherein the CY and FLU are administered daily for three consecutive days, or wherein the dose of CY is at about 500 mg/m² and the dose of FLU is at about 30 mg/m².

38. The use of any one of claims 31-37, wherein the BCMA directed CAR comprises:
(i) a variable heavy chain (VH) and a variable light chain (VL), wherein:
(a) said VH comprises: a heavy chain complementary determining region 1 (H-CDR1) with at least 80% sequence identity to SEQ ID NO: 8 (GFTFSRYW), a heavy chain complementary determining region 2 (H-CDR2) with at least 80% sequence identity to SEQ ID NO: 9 (INPSSSTI), and a heavy chain complementary determining region 3 (H-CDR3) with at least 80% sequence identity to SEQ ID NO: 10 (ASLYYDYGDAYDY); and
(b) a variable light chain (VL), said VL comprising: a light chain complementary determining region 1 (L-CDR1) with at least 80% sequence identity to SEQ ID NO: 11 (QSVESN), a light chain complementary determining region 2 (L-CDR2) with at least 80% sequence identity to SEQ ID NO: 12 (SAS), and a light chain complementary determining region 3 (L-CDR3) with at least 80% sequence identity to SEQ ID NO: 13 (QQYNNYPLT); or
(ii) an amino acid sequence that is of at least about 99%, 98%, 96%, 95%, 90%, 85%, or 80% identity to SEQ ID NO: 7.

39. A method of treating a subject diagnosed with multiple myeloma (MM), wherein the method comprises:
(i) administering to the subject at least a first cycle of an adoptive cell therapy product, with the first cycle comprising at least a first dose at a first frequency of the adoptive cell therapy product administered in a first effective amount, and with an option of administering one or more additional cycles with one or more additional doses at the same first frequency or a different frequency in a second effective amount, during a course of treatment over a period of time; and
(ii) administering to the subject weekly doses of an effective amount of an anti-CD38 monoclonal antibody or an anti-SLAMF7 monoclonal antibody for about 6-10 weeks, wherein a first weekly dose of the anti-CD38 monoclonal antibody or the anti-SLAMF7 monoclonal antibody precedes step (i) by about 8 to 12 days;
wherein the MM tumor progress or relapse is prevented or reduced in the subject after the treatment using the adoptive cell therapy product; and
wherein the adoptive cell therapy comprises engineered natural killer (NK) lineage cell comprising CD38 knockout, expressing an exogenous CD16, an IL15RF and a BCMA directed CAR.

40. The method of claim 39, wherein the subject
(i) has a refractory or a relapsed MM, and/or
(ii) has been treated with one or more lines of therapy for MM. comprising:
(a) a chemotherapy, an immunochemotherapy, hematopoietic stem cell transplantation, chimeric antigen receptor (CAR) T-cell therapy, or any combination thereof;
(b) a proteasome inhibitor, an anti-CD38 antibody, an anti-SLAMF7 antibody, an immunomodulatory drug, a stem-cell transplantation (SCT), or any combination thereof, or
(c) bortezomib, carfilzomib, ixazomib, daratumumab, isatuximab, elotuzumab, thalidomide, lenalidomide, pomalidomide, or any combination thereof.

41. The method of claim 39, wherein the step (ii) further comprises administering bi-weekly doses of an effective amount of the same anti-CD38 monoclonal antibody or anti-SLAMF7 monoclonal antibody for another 6-10 weeks after completing the weekly doses.

42. The method of claim 39, wherein the anti-CD38 monoclonal antibody is daratumumab and/or wherein the anti-SLAMF7 monoclonal antibody is elotuzumab.

43. The method of claim 39, further comprising administering to the subject a daily dose of one or more chemotherapeutic agents for three consecutive days, wherein the duration between the last daily dose administration of the one or more chemotherapeutic agents and a first weekly dose of the adoptive cell therapy product is about 40-84 hours.

44. The method of claim 43, wherein the one or more chemotherapeutic agents comprise cyclophosphamide (CY) and fludarabine (FLU), and optionally wherein the daily dose of CY is at about 500 mg/m² and the daily dose of FLU is at about 30 mg/m².

45. The method of claim 39, wherein the method: (a) does not require CY/FLU-based lympho-conditioning; or (b) requires a minimal need of CY/FLU-based lympho-conditioning; and wherein the step (ii) of the method comprises administering to the subject weekly doses of an effective amount of the anti-CD38 monoclonal antibody.

46. The method of claim 39, wherein the method does not require IL2 cytokine support to the subject during the course of treatment.

47. The method of claim 39, wherein the engineered NK lineage cell is derived from an engineered induced pluripotent stem cell (iPSC) comprising CD38 knockout, a polynucleotide encoding an exogenous CD16, and a polynucleotide encoding IL15RF.

48. The method of claim 39, wherein the effective amount of the adoptive cell therapy product in a dose is about 5×10⁷ cells/dose to 3×10⁹ cells/dose.

49. The method of claim 48, wherein the effective amount of the adoptive cell therapy product in each dose is about 1×10⁸, 3×10⁸, 10×10⁸, or about 1.5×10⁹ cells.

50. The method of claim 42, wherein:
(i) the daratumumab is in an effective amount of about 15 mg/kg to about 17 mg/kg; and/or
(ii) the elotuzumab is in an effective amount of about 9 mg/kg to about 11 mg/kg.

51. The method of claim 42, wherein:
(i) the effective amount of daratumumab is about 16 mg/kg; and
(ii) the effective amount of elotuzumab is about 10 mg/kg.

52. The method of claim 39, further comprising assessing disease response after a first cycle of administration of the adoptive cell therapy product.

53. The method of claim 52, wherein assessing disease response comprises assaying a bone marrow biopsy, a peripheral blood sample, and a urine sample from the subject for complete response or partial response based on criteria comprising leukemic blast count, absolute neutrophil count and/or platelet count, wherein:
(i) the complete response comprises:
(a) a negative immunofixation on serum from the subject, as compared to serum immunofixation prior to the course of treatment;
(b) a negative immunofixation on urine from the subject, as compared to urine immunofixation prior to the course of treatment;
(c) elimination of soft tissue plasmacytomas, as compared to plasmacytomas prior to the course of treatment; and/or
(d) <5% plasma cells in bone marrow, as compared to plasma cells in bone marrow prior to the course of treatment; and/or
(ii) the partial response comprises:
(a) ≥25% but ≤49% reduction of serum M-protein level, as compared to serum M-protein prior to the course of treatment; and/or
(b) reduction in 24-h urine M-protein level by about 50-89%, as compared to urine M-protein prior to the course of treatment.

54. The method of any one of claims 39-53, wherein the administering of the adoptive cell therapy product is (i) via intravenous infusion, and/or (ii) at a site of an outpatient setting; and/or
wherein each dose of the adoptive cell therapy product is cryopreserved, and then thawed prior to administering.

55. The method of any one of claims 39-54, wherein the BCMA directed CAR comprises:
(i) a variable heavy chain (VH) and a variable light chain (VL), wherein:
(a) said VH comprises: a heavy chain complementary determining region 1 (H-CDR1) with at least 80% sequence identity to SEQ ID NO: 8 (GFTFSRYW), a heavy chain complementary determining region 2 (H-CDR2) with at least 80% sequence identity to SEQ ID NO: 9 (INPSSSTI), and a heavy chain complementary determining region 3 (H-CDR3) with at least 80% sequence identity to SEQ ID NO: 10 (ASLYYDYGDAYDY); and
(b) a variable light chain (VL), said VL comprising: a light chain complementary determining region 1 (L-CDR1) with at least 80% sequence identity to SEQ ID NO: 11 (QSVESN), a light chain complementary determining region 2 (L-CDR2) with at least 80% sequence identity to SEQ ID NO: 12 (SAS), and a light chain complementary determining region 3 (L-CDR3) with at least 80% sequence identity to SEQ ID NO: 13 (QQYNNYPLT); or
(ii) an amino acid sequence that is of at least about 99%, 98%, 96%, 95%, 90%, 85%, or 80% identity to SEQ ID NO: 7.

56. A method of a multi-dose targeted adoptive cell therapy in a subject in need thereof comprising:
(i) weekly administration to the subject of an effective amount of the targeted adoptive cell therapy product for a course of treatment of about three weeks, wherein the product comprises: an engineered immune cell expressing CD16, IL15RF, and a BCMA directed CAR, and wherein the engineered immune cell is CD38 negative; and
(ii) detecting and comparing one or more of the following at different given time points following administration of a first dose of the adoptive cell therapy:
(a) the presence of the engineered immune cell in bone marrow of the subject;
(b) the presence of the engineered immune cell in a tumor of the subject;
(c) protein markers of disease in serum of the subject;
(d) cytokines in a peripheral blood sample from the subject; and
(e) circulating tumor DNA in a peripheral blood sample from the subject, wherein any of (a)-(e) is useful for assessing tumor burden, tumor immunobiology, and/or tumor therapy response, thereby determining efficacy of the multi-dose targeted adoptive cell therapy.

57. The method of claim 56, wherein the subject has multiple myeloma (MM).

58. The method of claim 56, wherein the effective amount of the adoptive cell therapy product is about 5×10⁷ cells/dose to 1.5×10⁹ cells/dose.

59. The method of claim 56, further comprising weekly administration to the subject of an effective amount of an anti-CD38 monoclonal antibody, or an anti-SLAMF7 monoclonal antibody, wherein a first dose of the anti-CD38 monoclonal antibody or an anti-SLAMF7 monoclonal antibody precedes step (i) by about 10 days.

60. The method of claim 59, wherein the anti-CD38 monoclonal antibody is daratumumab and/or the anti-SLAMF7 monoclonal antibody is elotuzumab, and/or the anti-CD20 monoclonal antibody is rituximab.

61. The method of any one of claims 56-60, wherein the BCMA directed CAR comprises:
(i) a variable heavy chain (VH) and a variable light chain (VL), wherein:
(a) said VH comprises: a heavy chain complementary determining region 1 (H-CDR1) with at least 80% sequence identity to SEQ ID NO: 8 (GFTFSRYW), a heavy chain complementary determining region 2 (H-CDR2) with at least 80% sequence identity to SEQ ID NO: 9 (INPSSSTI), and a heavy chain complementary determining region 3 (H-CDR3) with at least 80% sequence identity to SEQ ID NO: 10 (ASLYYDYGDAYDY); and
(b) a variable light chain (VL), said VL comprising: a light chain complementary determining region 1 (L-CDR1) with at least 80% sequence identity to SEQ ID NO: 11 (QSVESN), a light chain complementary determining region 2 (L-CDR2) with at least 80% sequence identity to SEQ ID NO: 12 (SAS), and a light chain complementary determining region 3 (L-CDR3) with at least 80% sequence identity to SEQ ID NO: 13 (QQYNNYPLT); or
(ii) an amino acid sequence that is of at least about 99%, 98%, 96%, 95%, 90%, 85%, or 80% identity to SEQ ID NO: 7.

62. A method of retreatment following one or more earlier line(s) of treatment of MM according to the method of any one of claims 1-21 or 39-61, wherein the one or more early lines of therapy for MM comprise:
(a) a chemotherapy, an immunochemotherapy, hematopoietic stem cell transplantation, chimeric antigen receptor (CAR) T-cell therapy, or any combination thereof;
(b) a proteasome inhibitor, an anti-CD38 antibody, an anti-SLAMF7 antibody, an immunomodulatory drug, a stem-cell transplantation (SCT), or any combination thereof,
(c) bortezomib, carfilzomib, ixazomib, daratumumab, isatuximab, elotuzumab, thalidomide, lenalidomide, pomalidomide, or any combination thereof, and/or
(d) idecabtagene vicleucel (bb2121, ide-cel), a BCMA-directed autologous CAR T-cell therapy.
